SEQUENCE LISTING (53 sequences)

SEQ ID NO: 1 - protein, 108 aa, artificial
  parental variable kappa chain domain
DIQMTQSPSSLSASVGDRVTITCRASQSISSYLAWYQQKPGKAPKLLIYAASSLQSGVPSRFSGSGSGTDFTLTISSLQPEDFATYYCQQYSSYPFTFGQGTKVEIKR

SEQ ID NO: 2 - protein, 110 aa, artificial
  parental variable lambda chain domain
QSVLTQPPSVSGAPGQRVTISCSGSSSNIGSNTVNWYQQLPGTAPKLLIYGNNNRPSGVPDRFSGSKSGTSASLAITGLQSEDEADYYCAAWDDSLNGVVFGGGTKLTVL

SEQ ID NO: 3 - protein, 116 aa, artificial
  parental variable heavy chain domain
EVQLVESGGGLVQPGGSLRLSCAASGFTFSSYAMSWVRQAPGKGLEWVGNISGSGGSTYYADSVKGRFTISRDNSKNTLYLQMNSLRAEDTAVYYCARASGYFDYWGQGTLVTVSS

SEQ ID NO: 4 - protein, 108 aa, artificial
  variable light chain domain variant
DTQMTQSPSSLSASVGDRVTITCRASQSISSYLAWYQQKPGKAPKLLIYAASSLQSGVPSRFSGSGSGTDFTLTISSLQPEDFATYYCQQYSSYPFTFGQGTKVEIKR

SEQ ID NO: 5 - protein, 116 aa, artificial
  variable heavy chain domain variant
EVQLVESGGGLVQPGGSLRLSCAASGFTFSSYAMSWVRQAPGKGLEWVGNISGSGGSTYYADSVKGRFTISRDNSKNTLYLQMNSLRAEDTAVYYCARDSGYFDYWGQGTLVTVSS

SEQ ID NO: 6 - protein, 108 aa, artificial
  variable light chain domain variant
DTQMTQSPSSLSASVGDRVTITCRASQSISSYLNWYQQKPGKAPKLLIYAASSLQSGVPSRFSGSGSGTDFTLTISSLQPEDFATYYCQQYSSYPFTFGQGTKVEIKR

SEQ ID NO: 7 - protein, 116 aa, artificial
  variable heavy chain domain variant
EVQLVESGGGLVQPGGSLRLSCAASGFTFSSYAMSWVRQAPGKGLEWVGNISGSGGSTYYADSVKGRFTISRDNSKNTLYLQMNSLRAEDTAVYYCARDSGYMDYWGQGTLVTVSS

SEQ ID NO: 8 - protein, 116 aa, artificial
  variable heavy chain domain variant
ETQLVESGGGLVQPGGSLRLSCAASGFTFSSYAMSWVRQAPGKGLEWVGNISGSGGSTYYADSVKGRFTISRDNSKNTLYLQMNSLRAEDTAVYYCARDSGYFDYWGQGTLVTVSS

SEQ ID NO: 9 - protein, 108 aa, artificial
  variable light chain domain variant
DTQMTQSPSSLSASVGDRVTITCRASQSISSYLAWYQQKPGKAPKLLIYAASSLQSGVPSRFSGSGSGTDFTLTISSLQPEDFATYYCQQYSSYPYTFGQGTKVEIKR

SEQ ID NO: 10 - protein, 116 aa, artificial
  variable heavy chain domain variant
EVQLVESGGGLVQPGGSLRLSCAASGFTFSSNYMSWVRQAPGKGLEWVGNISGSGGSTYYADSVKGRFTISRDNSKNTLYLQMNSLRAEDTAVYYCARDSGYFDIWGQGTLVTVSS

SEQ ID NO: 11 - protein, 116 aa, artificial
  variable heavy chain domain variant
EVQLVESGGGLVQPGGSLRLSCAASGFTFSSYAMSWVRQAPGKGLEWVGNISGSGGSTYYADSVKGRFTISRDNSKNTLYLQMNSLRAEDTAVYYCARDSGYFDIWGQGTLVTVSS

SEQ ID NO: 12 - protein, 108 aa, artificial
  variable light chain domain variant
DIQMTQSPSSLSASVGDRVTITCRASQSISSYLAWYQQKPGKAPKLLIYAASSLQSGVPSRFSGSGSGTDFTLTISSLQPEDFATYYCQQYSSYPYTFGQGTKVEIKR

SEQ ID NO: 13 - protein, 116 aa, artificial
  variable heavy chain domain variant
EVQLVESGGGLVQPGGSLRLSCAASGFTFSSYAMSWIRQAPGKGLEWIGQISGSGGSTYYNDSVKGRFTISRDNSKNTLYLQMNSLRAEDTAVYYCARDSGYFDIWGQGTLVTVSS

SEQ ID NO: 14 - protein, 116 aa, artificial
  variable heavy chain domain variant
EVQLVESGGGLVQPGGSLRLSCAASGFTFSSYAMSWVRQAPGKGLEWVGQISGSGGSTYYNDSVKGRFTISRDNSKNTLYLQMNSLRAEDTAVYYCARDSGYFDIWGQGTLVTVSS

SEQ ID NO: 15 - protein, 116 aa, artificial
  variable heavy chain domain variant
EVQLVESGGGLVQPGGSLRLSCAASGFTFSSYAMSWIRQAPGKGLEWVGQISGSGGSTYYNDSVKGRFTISRDNSKNTLYLQMNSLRAEDTAVYYCARDSGYFDIWGQGTLVTVSS

SEQ ID NO: 16 - protein, 116 aa, artificial
  variable heavy chain domain variant
EVQLVESGGGLVQPGGSLRLSCAASGFTFSSYAMSWVRQAPGKGLEWIGQISGSGGSTYYNDSVKGRFTISRDNSKNTLYLQMNSLRAEDTAVYYCARDSGYFDIWGQGTLVTVSS

SEQ ID NO: 17 - protein, 116 aa, artificial
  variable heavy chain domain variant
EVQLVESGGGLVQPGGSLRLSCAASGFTFSSYAMSWVRQAPGKGLEWMGQISGSGGSTYYNDSVKGRFTISRDNSKNTLYLQMNSLRAEDTAVYYCARDSGYFDIWGQGTLVTVSS

SEQ ID NO: 18 - protein, 108 aa, artificial
  variable light chain domain variant
DTQMTQSPSSLSASVGDRVTITCRASQSISSYLAWYQQKPGKAPKLLIYAASSLYSGVPSRFSGSGSGTDFTLTISSLQPEDFATYYCQQYSSYPYTFGQGTKVEIKR

SEQ ID NO: 19 - protein, 110 aa, artificial
  variable light chain domain variant
SSVLTQPPSVSGAPGQRVTISCSGSSSNIGSNTVNWYQQLPGTAPKLLIYGNNNRPSGVPDRFSGSKSGTSASLAITGLQSEDEADYYCAAWDDSLNGVVFGGGTKLTVL

SEQ ID NO: 20 - protein, 116 aa, artificial
  variable heavy chain domain variant
EVQLVESGGGLVQPGGSLRLSCAASGFTFSSYAMSWVRQAPGKGLEWVGTISGSGGSTYYADNVLGRFTISRDNSKNTLYLQMNSLRAEDTAVYYCARASGYFDYWGQGTLVTVSS

SEQ ID NO: 21 - protein, 117 aa, artificial
  variable heavy chain domain variant
EVQLVESGGGLVQPGGSLRLSCAASGFTFSSNYMSWVRQAPGKGLEWVGTISGSGGSTYYADNVLGRFTISRDNSKNTLYLQMNSLRAEDTAVYYCARDSSGYFDYWGQGTLVTVSS

SEQ ID NO: 22 - protein, 116 aa, artificial
  variable heavy chain domain variant
EVQLVESGGGLVQPGGSLRLSCAASGFTFSSYAMSWVRQAPGKGLEWVGTISGSGGSTYYADNVLGRFTISRDNSKNTLYLQMNSLRAEDTAVYYCARDSGYFDYWGQGTLVTVSS

SEQ ID NO: 23 - protein, 117 aa, artificial
  variable heavy chain domain variant
EVQLVESGGGLVQPGGSLRLSCAASGFTFSSNYMSWVRQAPGKGLEWVGTISGSGGSTYYADNVLGRFTISRDNSKNTLYLQMNSLRAEDTAVYYCARTSSGYFDYWGQGTLVTVSS

SEQ ID NO: 24 - protein, 110 aa, artificial
  variable light chain domain variant
SSVLTQPPSVSGAPGQRVTISCSGSSSNIGSNTVNWYQQLPGTAPKLLIYGNNNRPSGVPDRFSGSKSGTSASLAITGLQSEDEADYYCAAWDDSLNGYVFGGGTKLTVL

SEQ ID NO: 25 - protein, 110 aa, artificial
  variable light chain domain variant
SSVLTQPPSVSGAPGQRVTISCSGSSSNIGSNTVNWYQQLPGTAPKLLIYGNNNRPSGVPDRFSGSKSGTSASLAITGLQSEDEADYYCAAWDDSLNGYVFGTGTKLTVL

SEQ ID NO: 26 - protein, 110 aa, artificial
  variable light chain domain variant
SSVLTQPPSVSGAPGQRVTISCSGSSSNIGSNTVSWYQQLPGTAPKLLIYGNNNRPSGVPDRFSGSKSGTSASLAITGLQSEDEADYYCAAWDDSLNGYVFGGGTKLTVL

SEQ ID NO: 27 - protein, 108 aa, artificial
  variable light chain domain variant
AIQMTQSPSSLSASVGDRVTITCRASQSISSYLAWYQQKPGKAPKLLIYAASSLYSGVPSRFSGSGSGTDFTLTISSLQPEDFATYYCQQYSSLPYTFGQGTKVEIKR

SEQ ID NO: 28 - protein, 116 aa, artificial
  variable heavy chain domain variant
EAQLVESGGGLVQPGGSLRLSCAASGFTFSSYAMSWIRQAPGKGLEWIGQISGSGGSTYYNDSVKGRFTISRDNSKNTLYLQMNSLRPEDTAVYYCARDSGYFDIWGQGTLVTVSS

SEQ ID NO: 29 - protein, 246 aa, Homo sapiens
QVQLQQSGADLKVPGASVKVSCKSSGYWFHDYAALALGRAPGKGLEWTGWINTNYGETNYAQKFLGGVTMTRDKSTSTGTELIRLGSDDTAVYYCARLIVSDRYGQGTMVTASSGGGGSSGGGGSGGSALAIQMTQSPSSLSASVGDRVTITCRASQGIRNDLGWYQQKPGKAPKLLIYAASSLQSGVPSRFSGSGSGTDFTLTISSLQPEDFATYYCQQYNSYHTFGQGTKVEIKRAAAHHHHHH

SEQ ID NO: 30 - protein, 147 aa, Homo sapiens
MDMGAHVHLLGLLLLWLPGARCAIQMTQSPSSLSASVGDRVTITCRASQGIRNDLGWYQQKPGKAPKLLIYAASSLQSGVPSRFSGSRSGTDFTLTISSLQPEDFATYYCLRDYNYSWTFGQGTKVEIKRTVAAPSVFIFPPSDEAW

SEQ ID NO: 31 - protein, 95 aa, Homo sapiens
AIQMTQSPSSLSASVGDRVTITCRASQGIRNDLGWYQQKPGKAPKLLIYAASSLQSGVPSRFSGSGSGTDFTLTISSLQPEDFATYYCLQDYNYP

SEQ ID NO: 32 - protein, 109 aa, Homo sapiens
AIQMTQSPSSLSASVGDRVTITCRASQGIRNDLGWYQQKPGKAPKLLIYAASSLQSGVPSRFSGSRSGTDFTLTISSLQPEDFATYYCLRDYNYSWTFGQGTKVEIKRT

SEQ ID NO: 33 - protein, 108 aa, Homo sapiens
AIQMTQSPSSLSASVGDRVTITCRASQGIRNDLGWYQQKPGKAPKRLIYAAFIWQSGVPSRFSGSGSGTEFTLTISSLQPEDFATYYCLQHNSFPYTFGQGTKLEVKR

SEQ ID NO: 34 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 35 - protein, 107 aa, Homo sapiens; identical to SEQ ID NO: 34

SEQ ID NO: 36 - protein, 130 aa, Homo sapiens
MDMRVPAQLLGLLLLWLPGARCAIQLTQSPSSLSASVGDRVTITCRASQGISSALAWYQQKPGKAPKLLIYDASSLESGVPSRFSGSGSGTDFTLTISSLQPEDFATYYCQQFNIFGGGTKVEIKRIRAR

SEQ ID NO: 37 - protein, 125 aa, Homo sapiens
PAQLLGLLLLWLPGARCAIQLTQSPSSLSASVGDRVTITCRASQGISSALAWYQQKPGKAPKLLIYDASSLESGVPSRFSGSGSGTDFTLTISSLQPEDFATYYCQQFNTYPLTFGGGTKVEIKR

SEQ ID NO: 38 - protein, 95 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 39 - protein, 108 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNTYPLTFGGGTKVEIKR

SEQ ID NO: 40 - protein, 106 aa, Homo sapiens
AIQLTQSPSSLSASVGDRVTITCRASQGITSRSAWYQQKPGKAPRLLIYGVSNLESGVPSRFSGSASGTDFTLTISSLQPEDFATYYCQQINNSPAFGQGTRLEIK

SEQ ID NO: 41 - protein, 128 aa, Homo sapiens
LGLLLLWLPGARCAIQMTQSPSSLSASVGDRVTITCRASQDIRNDLGWYQQQPGKAPKLLIYAASTLHTGVPSRFSGSRSGTTFTLTISGLQPEDFATYYCLQDYNYWTFGQGTRVEIKRTVAAPSVF

SEQ ID NO: 42 - protein, 108 aa, Homo sapiens
DTQMTQSPSSLSASVGDRVTITCRASQSISSYLNWYQQKPGKAPKLLIYYASYLQSGVPSRFSGSGSGTDFTLTISSLQPEDFATYYCQQTYTAPDTFGQGTKVEIKR

SEQ ID NO: 43 - protein, 109 aa, Homo sapiens
MAETTLTQSPSSLSASVGDRVTITCRASQSISSYLNWYQQKPGKAPKLLIYAASSLQSGVPSRFSGSGSGTDFTLTISSLQPEDFATYYCQQSYSTPHTFGQGTKVEIK

SEQ ID NO: 44 - protein, 108 aa, Homo sapiens
DTQLTQSPSFLSASVGDRVTITCRASQGISSYLAWYQQKPGKAPRLLIYATSTLQSGVPSRFSGSGSGTEFTLTISSLQPEDFATYYCQQLNSYPPYTFGQGTKLEIN

SEQ ID NO: 45 - protein, 108 aa, Homo sapiens
PLTSMTQSPSSLSASIGDRVTITCRASQSISIFLNWFQQRPGKAPKLLIYAASSLQGGVPSRFSGSGSGTDFTLTITSLQPEDFATYYCQQSFSIPWTFGQGTNVDIK

SEQ ID NO: 46 - protein, 214 aa, Homo sapiens
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 214 aa, Homo sapiens; identical to SEQ ID NO: 46

SEQ ID NO: 48 - protein, 214 aa, Homo sapiens; identical to SEQ ID NO: 46

SEQ ID NO: 49 - protein, 101 aa, Homo sapiens
VQCEAQLLESGGGLVQRGGSLRLSCAASGFTFSNYAMSWVRQAPGKGLEWVSTTSGSGASTYHADSVKGRFTISRDNSKNTLYLQMNSLRAEDTAVYYCAK

SEQ ID NO: 50 - protein, 101 aa, Homo sapiens
VQCEAQLLESGGGLVQPGGSLRLSCAASGFTFTTYAMSWVRQAPGKGLEWVSTITGGGGGTDYADSVKGRFTISRDNSKNTLYLQMNSLRAEDTAVYYCAK

SEQ ID NO: 51 - protein, 101 aa, Homo sapiens
VQCEVQLLESGGGLVQPGGSLRLSCAASGFTFSNFAMSWIRQAPGKGLEWVSTLSGGGGSTYYADSVKGRFTISRDNSKNTLYLQMNSLRAEDTAVYHCGK

SEQ ID NO: 52 - protein, 121 aa, Homo sapiens
EVQLVESGGGLVQPGGSLRLSCVASGFTFTSYAMIWVRQAPGKGLEWISTINDSGGRTYYADSVKGRFTVSRDNSKNTLYLQMNSLRAEDSAVYYCVNDKERDDGGWRDPWGQGTLVTVSS

SEQ ID NO: 53 - protein, 101 aa, Homo sapiens
VQCEVQLLESGGGLVQPGGSLRLSCAASGFTFSNYPMSWIRQAPGKGLEWVSTLSGSGVTTFYADSGKGRFTISRDNSKNTLYLQMSSLRADDTAVYYCAK

(19) United States
(12) Patent Application Publication
(10) Pub. No.: US 2014/0213459 A1
(43) Pub. Date: Jul. 31, 2014

(54) ANTIBODIES WITH IMPROVED FOLDING STABILITY

(71) Applicant: Roland Beckmann, Vienna (AT)

(72) Inventor: Roland Beckmann, Vienna (AT)

(21) Appl. No.: 14/122,874

(22) PCT Filed: May 29, 2012
(86) PCT No.: PCT/EP2012/002278

§ 371 Date: Mar. 21, 2014

(30) Foreign Application Priority Data
May 27, 2011 (JP) 11004371.8

(51) Int. Cl. C12N 15/10; G01N 33/483; G06F 19/16; C07K 16/00

(52) U.S. Cl. CPC C12N 15/1058; C07K 16/00; G01N 33/483; G06F 19/16
USPC 506/1; 530/387.3; 435/69.6; 436/87; 506/26; 506/12; 506/18; 703/1

(57) ABSTRACT

The present invention relates to methods for improving the folding stability of antibodies, to antibodies with improved folding stability, nucleic acid and vectors encoding such antibodies, and to uses of such antibodies, nucleic acid and vectors.

### FIELD OF THE INVENTION

The present invention relates to methods for improving the folding stability of antibodies, to antibodies with improved folding stability, nucleic acid and vectors encoding such antibodies, and to uses of such antibodies, nucleic acid and vectors.

### BACKGROUND OF THE INVENTION

This invention relates to a novel approach for the stabilization of antibodies.

The biophysical stability of monoclonal antibodies is an important determinant of their usefulness and commercial value for several reasons (reviewed, among others, by Garber and Demarest, 2007, and by Honegger, 2008). First, high biophysical stability can result in high antibody expression yield in recombinant systems. High expression yield can facilitate antibody selection and screening, for example by enhancing the display level of antibodies on bacteriophages and by enhancing the soluble yield of antibodies in small-scale E. coli cultures, and therefore lead to the discovery of better antibody molecules. High expression yield can also be important in making the manufacturing of commercially available monoclonal antibodies economically viable by allowing a sufficiently small production scale suitable for the application. In monoclonal antibodies intended for therapeutic applications, high biophysical stability can be important as it can be associated with high solubility, therefore enabling antibodies to be efficiently formulated at high concentrations into drugs. Also in therapeutic monoclonal antibodies, high biophysical stability can be important for avoiding antibody aggregation during various manufacturing steps (including expression, purification, acid-mediated virus-deactivation and formulation) and during storage. The avoidance of aggregation is not only important for maximizing the economic viability of an antibody drug production process but is also thought to play an important role in minimizing the potential immunogenicity of antibody drugs in patients. Finally, also in therapeutic monoclonal antibodies, high biophysical stability is important in achieving a long antibody half-life both in patients and in disease models.

In recognition of the importance of high biophysical stability of monoclonal antibodies, researchers have aimed to improve the biophysical stability of monoclonal antibodies for the past several years. Research efforts have aimed to stabilize antibody constant domains, isolated antibody variable domains (especially autonomous VH domains known as VHH domains, VH domain antibodies, nanobodies or monobodies; but also autonomous VL domains), as well as heterodimeric antibodies comprising one VH and one VL domain. The read-out employed in such stabilization work has included increased expression yield, increased solubility of the expressed antibodies, increased levels of display in phage display libraries, increased resistance to denaturant-induced unfolding and increased resistance to heat-induced unfolding (known as thermal stability). In order to obtain antibody variable regions with improved biophysical stability, a variety of approaches has been taken that can be categorized as follows.

Researchers have employed specific antibody selection conditions, such as phage display with heat- or denaturant-induced stress during panning, to select antibody clones with superior biophysical properties, including reduced aggregation (Jung et al., 1999; Jespers et al., 2004(A); Dudgeon et al., 2008).

Researchers have employed specific antibody screening conditions, such as E. coli expression in the presence of reducing agents or fluorescent antigens able to permeate into the periplasm, or heating of secreted antibody clones in microtiter plates during antigen-specific ELISA screening, to select antibody clones with superior biophysical properties, including faster folding and greater thermal stability (Ribnicky et al., 2007; Martineau and Betton, 1999; Demarest et al., 2006).

Heterodimeric VH-VL antibody fragments have been stabilized by the addition of various entities such as chemical cross-linkers, peptide linkers to create single-chain Fv and single-chain Fab fragments, interchain disulphide bonds to create disulphide-stabilized Fv fragments, and heterodimeric coiled coils to create helix-stabilized Fv fragments (reviewed by Arndt et al., 2001).

Researchers have mutated framework residues at the VH-VL interface of heterodimeric VH-VL antibody fragments to obtain antibodies with greater resistance to denaturant-induced unfolding (Tan et al., 1998).

Heterodimeric VH-VL antibody fragments have been stabilized by increasing the hydrophilicity of solvent-exposed framework region residues. In some cases this has been done by disrupting hydrophobic patches at the antibody variable/constant domain interface (Nieba et al., 1997).

Autonomous VH domains have been stabilized by mutating positions otherwise contributing to the light chain interface, thereby improving solubility of this region that is buried in heterodimeric VH-VL antibodies (Davies and Riechmann, 1994; Riechmann, 1996; Bathelemy et al., 2007).

Autonomous VH domains have been stabilized by engineering additional intradomain disulphide bonds within the framework region (Davies and Riechmann, 1996) or between CDRs (Tanha et al., 2001).

In autonomous human and camelid VH domains, the CDR3 has been engineered to compensate for the hydrophobicity of the former light chain interface and to obtain better solubility of these domains (Tanha et al., 2001; Jespers et al., 2004(B); Dottorini et al., 2004).

In autonomous VL domains, a position in CDR1 (residue 32) and two positions in CDR2 (residues 50 and 56) have been engineered to increase the stability of the isolated VL domains towards denaturant-induced unfolding and to improve the feasibility of their potential use as disulphide-free intrabodies (Steipe, 1994; Ohage et al., 1997; Proba et al., 1998; Ohage and Steipe, 1999); all numbering according to Kabat (Kabat and Wu, 1991).

Germline genes from which VH or VL antibody domains are derived have been identified and analysed, and their sequences compiled into databases (Lefranc et al., 1999; Retter et al., 2005), and family-specific key residues have been identified that are critical for the family-specific folding and side-chain-packing within the VH or within the VL domain (Ewert et al., 2003(A)). Then, by aligning germline genes, protein consensus sequences have been generated for variable domains that contain more of the family-specific key residues than variable domains derived from individual germline genes and as a result have potentially improved biophysical properties over variable domains derived from individual germline genes (Steipe et al., 1994; reviewed by Wörn and Plückthun, 2001). Resulting human variable domain consensus sequences have been used in the humanization of animal-derived monoclonal antibodies (for example, Carter et al., 1992) and in the construction of synthetic human antibody libraries (for example, Knappik et al., 2000).

Based on consensus sequences, human VH and VL germline families have been characterized, families with inferior or superior biophysical properties have been identified, and individual framework region residues responsible for the inferior or superior properties have been pin-pointed (for example, Ewert et al., 2003(A)). This has allowed researchers to generate antibodies with improved biophysical properties in several ways:

Human antibody clones of known specificity have been stabilized by human-to-human CDR grafting: Antigen-specific CDR loops and selected putative specificity-enhancing framework region residues from a donor clone derived from a human germline gene associated with inferior biophysical properties were transplanted onto a human acceptor framework associated with superior biophysical properties (Jung and Plückthun, 1997).

Human antibody clones of known specificity have been stabilized by framework-engineering: A set of framework region residues thought to be responsible for inferior properties of one germline family has been exchanged for a set of different framework region residues found in a germline family associated with superior properties, thereby improving the biophysical properties of the antibody clone while retaining most of the original framework region sequences and while retaining the specificity (Ewert et al., 2003(B)).

Based on the ranking of the biophysical properties of human germline family consensus genes in the context of VH-VL pairings, researchers have suggested that synthetic antibody libraries should be prepared in which only those germline families with a consensus that had shown superior biophysical properties in the VH-VL pairings (VH1, VH3 and VH5 as well as Vkappa1, Vkappa3 and Vlambda) should be represented (Ewert et al., 2003(A)).

Researchers have generated synthetic antibody libraries in which all germline families were represented, but all clones derived from a VH germline family associated with inferior biophysical properties (VH4) contained a point-mutation in the framework region designed to improve the biophysical properties of these clones (Rothe et al., 2008).

Researchers have generated synthetic libraries of VH-VL heterodimeric antibodies based on a single synthetic VH framework and a single synthetic VL framework (for example, Lee et al., 2004; Fellouse et al., 2007) or on a single synthetic VH framework and multiple synthetic VL frameworks (for example, Silacci et al., 2005) known for their favourable biophysical framework properties.

Efforts have been made to obtain naturally occurring and therefore potentially stable CDR conformations in synthetic libraries of single domain antibodies and VH-VL heterodimeric antibodies, in order to give the antibodies nature-like and good, albeit not especially improved, biophysical properties. To this end, some CDR positions that are known to be determinants of specific canonical CDR structures (Chothia et al., 1992; Tomlinson et al., 1995; Al-Lazikani et al., 1997) have been left undiversified or subjected to restricted diversification in many published synthetic antibody libraries, maintaining them as the dominant residue or residues most frequently found in the germline family context of the particular VH or VL domain on which the library is based. Among such positions that bear canonical-structure-determining CDR residues and that have been left undiversified or subjected to restricted diversification in published antibody libraries are positions 27, 29 and 34 in HCDR1, positions 52a, 54 and 55 in HCDR2, and positions 94 and 101 in HCDR3, as well as positions 90 and 95 in the LCDR3 of Vkappa domains (all numbering according to Kabat (Kabat and Wu, 1991)).

Also in efforts to obtain natural and potentially stable CDR conformations in synthetic libraries of single domain antibodies and VH-VL heterodimeric antibodies, other CDR residues buried within the VH domain or within the VL domain, which are naturally conserved independently of different specific canonical CDR structures, have been left undiversified or subjected to restricted diversification in some synthetic antibody libraries, maintaining them as the dominant amino acid most frequently found in nature (for example, VH position 51 in HCDR2 has been kept undiversified in several published synthetic human antibody libraries, bearing an invariant Ile).

In contrast to the attention the naturally conserved residues described above have received in synthetic antibody library designs, very little work has been done with VH-VL heterodimeric antibodies on engineering the many, widely divergent CDR residues, which are not buried within the VH domain or within the VL domain and are not determinants of any specific canonical structure, towards superior biophysical properties of the final heterodimeric antibody. Instead, in published library designs, these residues have usually been diversified with the aim of maximizing antigen binding. This has typically been done either by complete or near-complete diversification to all naturally occurring amino acids, or by diversification regimens that aimed to reflect a natural distribution of amino acids in a particular CDR position, or by restricted diversification that maximized representation of amino acids known to be statistically important to antigen binding whilst minimizing library complexity. Examples of diversification regimens employed by previous researchers for these positions include the following:
1) full representation of all 20 amino acids (for example, Silacci et al., 2005);
2) representation of all 19 amino acids except for the oxidizable Cys (for example, Hoet et al., 2006);
3) a representation of amino acids designed to be nature-like, using restricted diversity with preference given to naturally frequent residues for that position (reviewed by Persson, 2009);
4) a representation of amino acids designed to enhance potential binding properties of the selected antibodies, using restricted diversity with preference given to amino acids known to be statistically important for antigen binding, such as Tyr and Ser (Fellouse et al., 2005) or Tyr, Ser, Gly and Asp (Fellouse et al., 2006).

Except for a few exceptions (see below), very little work has been done with VH-VL heterodimeric antibodies in relation to engineering the many, widely divergent CDR residues, which are not buried within the VH domain or within the VL domain and are not determinants of any specific canonical structure, towards superior biophysical properties of the final heterodimeric antibody.

Although not in the context of biophysical stability, Ueda et al. (1995) have observed that residue 95 in CDR3 of human VH domains, which is neither canonical-structure-determining nor necessarily buried within the VH domain, has an impact on the affinity of the heavy chain towards light chains in the context of VH-VL pairings. The investigators speculated that this residue may play a role in determining the shape of the VH CDR3 loop and observed that VH domains with the flexible residue Gly in position 95 appeared to exhibit the highest average affinity for light chains.

In work aimed at selecting stabilized antibodies by phage display, Jung et al. (1999) selected a mutant of the single chain Fv fragment 4D5Flu, which carried the two point mutations His to Asn in position 27d of LCDR1 and Phe to Val in position 55 of LCDR2. The investigators found that this mutant was more highly expressed and its thermal stability in DSC measurements was increased from 62.3° C. to 66.2° C. in PBS. The investigators suggested that single mutants should be analyzed to delineate the effects of the two mutations.

In work aimed at stabilizing a human tetanus toxoid-specific Fab fragment, Demarest et al. (2006) have observed that mutating residue 50 in LCDR1 of a human Vkappa domain (which is neither canonical-structure-determining nor usually buried within the Vkappa domain) from the wild-type residue Trp to smaller residues His and Ala significantly increased the biophysical stability of a heterodimeric VH-VL antibody against tetanus toxoid. The investigators speculated that the large native residue Trp likely causes a steric clash with HCDR3 residues of the antibody clone under investigation.

However, despite the fact that many attempts have been made to provide stable antibody frameworks and/or to stabilize existing antibodies, so far these attempts have had limited success.

Thus, there was still a large unmet need to provide novel methods for the stabilization of antibodies and novel stable antibody frameworks for the generation of antibody libraries or for CDR-grafting and/or humanization approaches.

The solution for this problem that has been provided by the present invention, i.e. the modification of particular residues in the CDR regions and/or conserved framework residues, has so far not been achieved or suggested by the prior art.

### SUMMARY OF THE INVENTION

The present invention relates to a method for improving the folding stability of antibodies and to antibodies with improved folding stability.

In a first aspect, the present invention relates to a method for modifying a parental antibody variable domain comprising a variable heavy (VH) chain domain and a variable light (VL) chain domain, comprising the steps of:
(a) establishing a structural model of said parental antibody variable domain based on its amino acid sequence;
(b) identifying in the six CDR regions of the VH and VL chain domains one or more CDR amino acid residues, which are buried in the interface between the VH domain and the VL domain, and which are not determinants of a specific canonical structure;
(c) replacing at least one of the amino acid residues identified in step (b) by a different amino acid residue to generate one or more antibody variable domain variants;
(d) optionally replacing in step (c) one or more additional amino acid residues in the CDR regions and/or in the framework regions of said parental antibody variable domain.

In a second aspect, the present invention relates to a method for modifying a parental antibody variable domain, comprising the step of:
(i) making or causing in a parental Vkappa1 antibody variable domain one or more of the following changes:
   (a) in the CDR regions:
      (aa) at position L55 a change to an amino acid selected from Y, H, and W, particularly to Y;
      (ab) at position L94 a change to an amino acid selected from F, H, I, K, L, M, R, T, V, and Y, particularly L;
      (ac) at position L96 a change to an amino acid selected from F and Y, particularly Y;
      (ad) at position L32 a change to an amino acid selected from D, F, K, N, Q, S, and Y;
      (ae) at position L34 a change to an amino acid selected from A, S, and T, particularly A and S, particularly A;
      (af) at position L91 a change to an amino acid selected from A, G, S, and Y, particularly Y; and/or
   (b) in the framework regions:
      (ba) at position L1 a change to amino acid A;
      (bb) at position L2 a change to amino acid T; and/or
      (bc) at position L70 a change to amino acid E; or
(ii) making or causing in a parental Vlambda1 antibody variable domain one or more of the following changes:
   (a) in the CDR regions:
      (aa) at position L34 a change to an amino acid selected from G and S, particularly S;
      (ab) at position L96 a change to an amino acid selected from F and Y, particularly to Y; and/or
      (ac) at position L100 a change to amino acid T; and/or
(iii) making or causing in a parental VH3 antibody variable domain one or more of the following changes:
   (a) in the CDR regions:
      (aa) at position H50 a change to an amino acid selected from Q, S and T, particularly S and T, particularly S, when said VH3 antibody variable domain is combined with a Vkappa antibody variable domain;
      (ab) at position H60 a change to amino acid N;
      (ac) at position H63 a change to an amino acid selected from V, I, and F;
      (ad) at position H64 a change to amino acid L;
      (ae) at position H95 a change to amino acid selected from D, N and T, particularly to D;
      (af) at position H102 a change to an amino acid selected from I and V;
      (ag) at position H28 a change to amino acid P;
      (ah) at position H33 a change to amino acid A;
      (ai) at position H52 a change to an amino acid selected from D and S, particularly to D;
      (aj) at position H(103 minus 5) a change to amino acid G;
      (aj) one or two changes at positions H50 and H95 in order to create a salt bridge, particularly the following salt bridges: H50:R/H95:E; and H50:H/H95:E;
      (ak) one or two changes at positions H33 and H95 in order to create a salt bridge, particularly the following salt bridges: H33:R/H95:E; H33:R/H95:D; H33:H/H95:D; and H33:D/H95:H; and/or
   (b) in the framework regions:
      (ba) at position H2 a change to an amino acid selected from A and G;
      (bb) at position H37 a change to amino acid I;
      (bc) at position H48 a change to amino acid I; and/or
      (bd) at position H49 a change to amino acid G.

In a third aspect, the present invention relates to an antibody variable domain comprising at least one VL or VH domain selected from the group of:
(i) a Vkappa1 antibody variable domain based on the antibody variable domain of SEQ ID No. 1, comprising one or more of the following changes:
(A) a single amino acid exchange L2:I to L2:T; or
(B) at least two amino acid changes independently selected from the following group:
(a) in the CDR regions:
   (aa) L55:Q to L55:Y;
   (ab) L94:T to L94:L; and/or
   (ac) L96:L to L96:Y; and/or
(b) in the framework regions:
   (ba) L1:D to L1:A;
   (bb) L2:I to L2:T; and/or
   (bc) L70:D to L70:E;
and optionally comprising up to 3 additional changes in the framework regions FR1 to FR3 different from those of (i)(A) and/or (B);
provided that the antibody variable domains having the following accession numbers are excluded: AJ704539, U43767, 4762, 40096, 21224, CS483741, CS483744, U86790, X72459, 4753, 19244, AY043163, L26891, DQ184511, AY686924, 4806, DQ535161, 1S78_C, 1S78_E, and 1L7I_L (accession numbers according to Abysis (http://www.bioinf.org.uk/abysis/index.html); see Table 2 after Examples);
(ii) a Vlambda1 antibody variable domain based on the antibody variable domain of SEQ ID No. 2, comprising the following combination of changes:
   (a) in the CDR regions:
      (aa) L34:N to L34:S; and
      (ab) L96:V to L96:Y or L96:V to L96:F;
and optionally further comprising up to 3 additional changes in the framework regions FR1 to FR3 different from those of (ii)(a);
(iii) a VH3 antibody variable domain based on the antibody variable domain of SEQ ID No. 3, comprising one or more of the following changes:
(A) a single amino acid exchange selected from the following group:
   (a) in the CDR regions:
      (aa) H50V: to H50:T;
      (ab) H60A: to H60:N;
      (ac) H63V: to H63:I
      (ad) H63V: to H63:F; and
      (ae) H64:K to H64:L, provided that H:63 is not D;
(B) at least two amino acid changes independently selected from the following group:
   (a) in the CDR regions:
      (aa) H50V: to H50:Q;
      (ab) H50V: to H50:T;
      (ac) H60A: to H60:N;
      (ad) H63V: to H63:I
      (ae) H63V: to H63:F;
      (af) H64:K to H64:L, provided that H:63 is not D; and
      (ag) H95:D to H95: N; and/or
   (b) in the framework regions:
      (ba) H2:V to H2:A;
      (bb) H37:V to H37:I;
      (bc) H48:V to H48:I; and/or
      (bd) H49:S to H49:G;
   in both (A) and (B) provided that the antibody variable domains having the following accession numbers are excluded: AM082547, AM082383, AM080583, AF471288, and AM082399 (accession numbers according to Abysis (http://www.bioinf.org.uk/abysis/index.html); see Table 2 after Examples).

In a fourth aspect, the present invention relates to a method for modifying an antibody variable domain, comprising the step of:
(a) making or causing in a Vkappa1 antibody variable domain having a sequence identity in the framework regions FR1 to FR3 of at least 90% to the antibody variable domain of SEQ ID No. 1 one or more of the following changes:
   (a) in the CDR regions:
      (aa) L55:Q to L55:(Y,H,W), particularly L55:Y;
      (ab) L94:T to L94:(F, H, I, K, L, M, R, T, V, Y), particularly L94:L; and/or
      (ac) L96:L to L96:(F,Y);
      (ad) L32:Y to L32(D, F, K, N, Q, S); and/or
   (b) in the framework regions:
      (ba) L1:D to L1:(A,D), particularly L1:A;
      (bb) L2:I to L2:T; and/or
      (bc) L70:D to L70:E;
(ii) making or causing in a Vlambda1 antibody variable domain having a sequence identity in the framework regions FR1 to FR3 of at least 90% to the antibody variable domain of SEQ ID No. 2 one or more of the following changes:
   (a) in the CDR regions:
      (aa) L34:N to L34:S;
      (ab) L96:V to L96:Y; and/or
      (ac) L96:V to L96:F; and/or
(iii) making or causing in a VH3 antibody variable domain having a sequence identity in the framework regions FR1 to FR3 of at least 90% to the antibody variable domain of SEQ ID No. 3 one or more of the following changes:
   (a) in the CDR regions:
      (aa) H50:V to H50:Q;
      (ab) H50:V to H50:T;
      (ac) H60:A to H60:V;
      (ad) H63:V to H63:I
      (ae) H63:V to H63:F
      (af) H63:V to H63:Q and/or
      (ag) H64:K to H64:L, provided that H:63 is not D; and
      (ah) H95:D to H95: N;
      (ai) H50:V to H50:S, particularly when said VH3 antibody variable domain is combined with a Vkappa antibody variable domain;
      (aj) H28:T to H28:P;
      (ak) H52:S to H52:D;
      (al) H(103-5):X to H(103-5):G;
      (am) H50/H95 to a salt bridge, particularly a salt bridge selected from: H50:R/H95:E; and H50:H/H95:E;
      (an) H33/H95 to a salt bridge, particularly a salt bridge selected from: H33:R/H95:E; H33:R/H95:D; H33:H/H95:D; and H33:D/H95:H; and/or
   (b) in the framework regions:
      (ba) H2:V to H2:A;
      (bb) H37:V to H37:I;
      (bc) H48:V to H48:I; and/or
      (bd) H49:S to H49:G.

In a fifth aspect, the present invention relates to the use of an antibody variable domain according to the present invention, or an antibody variable domain modified according to the present invention, in the construction of a diverse collection of antibody variable domains, comprising the step of:
(a) diversifying one or more amino acid positions in one or more CDR regions of said antibody variable domain, provided that
   (aa) none of the following CDR positions is diversified: Vkappa1: L96; Vlambda1: L96; VH3: H50 and H95; and the following CDR positions are each independently optionally diversified: Vkappa1: L55 and L94; VH3: H60, H63, and H64; or
   (ab) any of the following CDR positions is not diversified, if it carries one of the following amino acid residues: Vkappa1: L55:Y, L94:L, L96:Y; Vlambda1: L96:Y; VH3: H50:T, H60:N, H63:I, H64:L, and H95:D; or
   (ac) any of the following CDR positions is either not diversified, or it is diversified with a bias towards the following amino acid residues: Vkappa1: L55:Y, L94:L, L96:Y; Vlambda1: L96:Y; VH3: H50:T, H60:N, H63:I, H64:L, and H95:D; particularly wherein the listed amino acid residues is present to at least 30%, and more particularly to at least 50% in the diversification mixture; or
   (ad) any of the following CDR positions is either not diversified or diversified with the indicated limited diversity only: Vkappa1: L55:YHW, L94:FHIKLRY, L96:FY; Vlambda1: L96:FY; VH3: H50:QT, H60:HNRS, H63:VIF, H64:KL, and H95:DNT.

In a sixth aspect, the present invention relates to a method for construction of a diverse collection of antibody variable domains, comprising the step of (a) diversifying one or more amino acid positions in one or more CDR regions of an antibody variable domain according to claim 1, or an antibody variable domain modified according to the method of claim 2, provided that
(aa) none of the following CDR positions is diversified: Vkappa1: L96; Vlambda1: L96; VH3: H50 and H95; and the following CDR positions are each independently optionally diversified: Vkappa1: L55 and L94; VH3: H60, H63, and H64; or
(ab) any of the following CDR positions is not diversified, if it carries one of the following amino acid residues: Vkappa1: L55:Y, L94:L, L96:Y; Vlambda1: L96:Y; VH3: H50:T, H60:N, H63:I, H64:L, and H95:D; or
(ac) any of the following CDR positions is either not diversified, or it is diversified with a bias towards the following amino acid residues: Vkappa1: L55:Y, L94:L, L96:Y; Vlambda1: L96:Y; VH3: H50:T, H60:N, H63:I, H64:L, and H95:D; particularly wherein the listed amino acid residues is present to at least 30%, and more particularly to at least 50% in the diversification mixture; or
(ad) any of the following CDR positions is either not diversified or diversified with the indicated limited diversity only: Vkappa1: L55:YHW, L94:FHIKLRY, L96:FY; Vlambda1: L96:FY; VH3: H50:QT, H60:HNRS, H63:VIF, H64:KL, and H95:DNT.

In a seventh aspect, the present invention relates to a diverse collection of antibody variable domains, wherein said collection comprises one or more diverse collections of amino acid residues at one or more positions in one or more CDR regions, provided that
(aa) none of the following CDR positions is diversified: Vkappa1: L96; Vlambda1: L96; VH3: H50 and H95; and the following CDR positions are each independently optionally diversified: Vkappa1: L55 and L94; VH3: H60, H63, and H64; or
(ab) any of the following CDR positions is not diversified, if it carries one of the following amino acid residues: Vkappa1: L55:Y, L94:L, L96:Y; Vlambda1: L96:Y; VH3: H50:T, H60:N, H63:I, H64:L, and H95:D; or
(ac) any of the following CDR positions is either not diversified, or it is diversified with a bias towards the following amino acid residues: Vkappa1: L55:Y, L94:L, L96:Y; Vlambda1: L96:Y; VH3: H50:T, H60:N, H63:I, H64:L, and H95:D; particularly wherein the listed amino acid residues is present to at least 30%, and more particularly to at least 50% in the diversification mixture; or
(ad) any of the following CDR positions is either not diversified or diversified with the indicated limited diversity only: Vkappa1: L55:YHW, L94:FHIKLRY, L96:FY; Vlambda1: L96:FY; VH3: H50:QT, H60:HNRS, H63:VIF, H64:KL, and H95:DNT;
wherein the antibody variable domain is selected from the group of:
(i) a Vkappa1 antibody variable domain having a sequence identity in the framework regions FR1 to FR3 of at least 90% to the antibody variable domain of SEQ ID No. 1, optionally comprising one or more of the following changes:
   (b) in the framework regions:
      (ba) L1:D to L1:A;
      (bb) L2:I to L2:T; and/or
      (bc) L70:D to L70:E;
(ii) a Vlambda1 antibody variable domain having a sequence identity in the framework regions FR1 to FR3 of at least 90% to the antibody variable domain of SEQ ID No. 2; and/or
(ii) a VH3 antibody variable domain having a sequence identity in the framework regions FR1 to FR3 of at least 90% to the antibody variable domain of SEQ ID No. 3, optionally comprising one or more of the following changes:
   (b) in the framework regions:
      (ba) H2:V to H2:A;
      (bb) H37:V to H37:I;
      (bc) H48:V to H48:I; and/or
      (bd) H49:S to H49:G.

In an eighth aspect, the present invention relates to an antibody that has a melting temperature of significantly above 92° C. when analyzed by differential scanning calorimetry in pure 1× phosphate buffered saline pH 7.4, (containing 1.06 mM KH₂PO₄, 2.97 mM Na₂HPO₄×7H₂O, 155.17 mM NaCl and no other supplements) using a scan-rate of 60° C. per hour, no gain and a scan range of 32° C. to 115° C.

In a ninth aspect, the present invention relates to nucleic acid sequence encoding the antibody or functional fragment thereof according to the present invention.

In a tenth aspect, the present invention relates to a vector comprising the nucleic acid sequence according to the present invention.

In an eleventh aspect, the present invention relates to a host cell comprising the nucleic acid sequence according to the present invention, or the vector according to the present invention.

In a twelfth aspect, the present invention relates to a method for generating the antibody or functional fragment thereof according to the present invention, comprising the step of expressing the nucleic acid sequence according to the present invention, or the vector according to the present invention, either in vitro of from an appropriate host cell, including the host cell according to the present invention.

In a thirteenth aspect, the present invention relates to pharmaceutical compositions comprising an antibody molecule or functional fragment thereof, and optionally a pharmaceutically acceptable carrier and/or excipient. The compositions may be formulated e.g. for once-a-day administration, twice-a-day administration, or three times a day administration.

### BRIEF DESCRIPTION OF THE DRAWINGS

FIG. 1 shows exemplary graphs of typical DSC scans: FIG. 1(A): scan at 250°/h; FIG. 1(B): Krd13.5 with scan speed at 60°/h.

FIG. 2 contains sequence information for the parental variable domains Vkappa1 (SEQ ID No. 1), Vlambda1 (SEQ ID No. 2), and VH3 (SEQ ID No. 3), incl. the number scheme used in the context of the present invention. Framework regions FR1 to FR4 are shaded in grey.

FIG. 3 shows the DSC scan of Fab fragment KRd15.6 with scan-rate 60° C./h.

### DETAILED DESCRIPTION OF THE INVENTION

The peculiarity of this invention compared to former approaches for stabilizing antibodies is the so far unknown effect of modifications to CDR residues and to highly conserved residues in the framework regions, which results in antibodies with unprecedented stabilities.

In a first aspect, the present invention relates to a method for modifying a parental antibody variable domain comprising a variable heavy (VH) chain domain and a variable light (VL) chain domain, comprising the steps of
(a) establishing a structural model of said parental antibody variable domain based on its amino acid sequence;
(b) identifying in the six CDR regions of the VH and VL chain domains one or more CDR amino acid residues, which are buried in the interface between the VH domain and the VL domain, and which are not determinants of a specific canonical structure;
(c) replacing at least one of the amino acid residues identified in step (b) by a different amino acid residue to generate one or more antibody variable domain variants;
(d) optionally replacing in step (c) one or more additional amino acid residues in the CDR regions and/or in the framework regions of said parental antibody variable domain.

As used herein, the term “antibody” refers to an immunoglobulin (Ig) molecule that is defined as a protein belonging to the class IgG, IgM, IgE, IgA, or IgD (or any subclass thereof), which includes all conventionally known antibodies and functional fragments thereof. A “functional fragment” of an antibody/immunoglobulin molecule hereby is defined as a fragment of an antibody/immunoglobulin molecule (e.g., a variable region of an IgG) that retains the antigen-binding region. An “antigen-binding region” of an antibody typically is found in one or more hypervariable region(s) (or complementarity-determining region, “CDR”) of an antibody molecule, i.e. the CDR-1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). A preferred class of antibody molecules for use in the present invention is IgG.

“Functional fragments” of the invention include the domain of a F(ab′)2 fragment, a Fab fragment, scFv or constructs comprising single immunoglobulin variable domains or single domain antibody polypeptides, e.g. single heavy chain variable domains or single light chain variable domains. The F(ab′)2 or Fab may be engineered to minimize or completely remove the intermolecular disulphide interactions that occur between the CH1 and CL domains.

An antibody may be derived from immunizing an animal, or from a recombinant antibody library, including an antibody library that is based on amino acid sequences that have been designed in silico and encoded by nucleic acids that are synthetically created. In silico design of an antibody sequence is achieved, for example, by analyzing a database of human sequences and devising a polypeptide sequence utilizing the data obtained therefrom. Methods for designing and obtaining in silico-created sequences are described, for example, in Knappik et al., J. Mol. Biol. (2000) 296:57; Krebs et al., J. Immunol. Methods. (2001) 254:67; and U.S. Pat. No. 6,300,064 issued to Knappik et al.

As used herein, a binding molecule is “specific to/for”, “specifically recognizes”, or “specifically binds to” a target, such as a target biomolecule (or an epitope of such biomolecule), when such binding molecule is able to discriminate between such target biomolecule and one or more reference molecule(s), since binding specificity is not an absolute, but a relative property. In its most general form (and when no defined reference is mentioned), “specific binding” is referring to the ability of the binding molecule to discriminate between the target biomolecule of interest and an unrelated biomolecule, as determined, for example, in accordance with a specificity assay methods known in the art. Such methods comprise, but are not limited to Western blots, ELISA, RIA, ECL, IRMA tests and peptide scans. For example, a standard ELISA assay can be carried out. The scoring may be carried out by standard colour development (e.g. secondary antibody with horseradish peroxide and tetramethyl benzidine with hydrogenperoxide). The reaction in certain wells is scored by the optical density, for example, at 450 nm. Typical background (=negative reaction) may be about 0.1 OD; typical positive reaction may be about 1 OD. This means the ratio between a positive and a negative score can be 10-fold or higher. Typically, determination of binding specificity is performed by using not a single reference biomolecule, but a set of about three to five unrelated biomolecules, such as milk powder, BSA, transferrin or the like.

In the context of the present invention, the term “about” or “approximately” means between 90% and 110% of a given value or range.

However, “specific binding” also may refer to the ability of a binding molecule to discriminate between the target biomolecule and one or more closely related biomolecule(s), which are used as reference points. Additionally, “specific binding” may relate to the ability of a binding molecule to discriminate between different parts of its target antigen, e.g. different domains, regions or epitopes of the target biomolecule, or between one or more key amino acid residues or stretches of amino acid residues of the target biomolecule.

In certain embodiments, the antibody or functional fragment of the present invention is selected from a single chain Fv fragment, a Fab fragment and an IgG.

Functional fragments according to the present invention may be Fv (Skerra, A. & Plückthün (1988). Assembly of a functional immunoglobulin Fv fragment in Escherichia coli. Science 240, 1038-1041), scFv (Bird, R. E., Hardman, K. D., Jacobson, J. W., Johnson, S., Kaufman, B. M., Lee, S. M., Lee, T., Pope, S. H., Riordan, G. S. & Whitlow, M. (1988). Single-chain antigen-binding proteins. Science 242, 423-426.; Huston, J. S., Levinson, D., Mudgett-Hunter, M., Tai, M. S., Novotny, J., Margolies, M. N., Ridge, R. J., Bruccoleri, R. E., Haber, E., Crea, R. & Oppermann, H. (1988). Protein engineering of antibody binding sites: recovery of specific activity in an anti-digoxin single-chain Fv analogue produced in Escherichia coli. Proc. Natl. Acad. Sci. USA 85, 5879-5883.), disulfide-linked Fv (Glockshuber, R., Malia, M., Pfitzinger, I. & Plückthun, A. (1992). A comparison of strategies to stabilize immunoglobulin Fv-fragments. Biochemistry 29, 1362-1367.; Brinkmann, U., Reiter, Y., Jung, S., Lee, B. & Pastan, I. (1993). A recombinant immunotoxin containing a disulfide-stabilized Fv fragment. Proc. Natl. Acad. Sci. U.S.A. 90, 7538-7542.), Fab, (Fab′) 2 fragments, single VH domains or other fragments well-known to the practitioner skilled in the art, which comprise at least one variable domain of an immunoglobulin or immunoglobulin fragment and have the ability to bind to a target.

In particular embodiments, steps (c) and optionally (d) are performed by modifying one or more nucleic acid sequences encoding the parental antibody variable domain.

In particular embodiments, the method of the present invention comprises the additional step of:
(e) expressing the one or more nucleic acid sequences encoding each of said one or more antibody variable domain variants.

In particular embodiments, the method comprises the additional steps of:
(f) comparing the stability of said one or more antibody variable domain variants with the stability of the parental antibody; and
(g) selecting an antibody variable domain variant with improved stability.

In particular embodiments, the method comprises the additional step of:
(h) repeating steps (c) to (g) one or more times by using the antibody variable domain variant selected in the previous step (g) as new parental antibody variable domain in step (c).

In particular embodiments, the invention relates to a method, wherein in step (c) or (d) at least one amino acid residue is changed from an amino acid being the consensus amino acid for that position in the family of antibody sequences the parental antibody variable domain belongs to a non-consensus amino acid.

In a second aspect, the present invention relates to a method for modifying a parental antibody variable domain, comprising the step of:
(i) making or causing in a parental Vkappa1 antibody variable domain one or more of the following changes:
   (a) in the CDR regions:
      (aa) at position L55 a change to an amino acid selected from Y, H, and W, particularly to Y;
      (ab) at position L94 a change to an amino acid selected from F, H, I, K, L, R, and Y, particularly to L; and/or
      (ac) at position L96 a change to an amino acid selected from F and Y, particularly Y; and/or
   (b) in the framework regions:
      (ba) at position L1 a change to amino acid A;
      (bb) at position L2 a change to amino acid T; and/or
      (bc) at position L70 a change to amino acid E; or
(ii) making or causing in a parental Vlambda1 antibody variable domain one or more of the following changes:
   (a) in the CDR regions:
      (aa) at position L34 a change to amino acid S;
      (ab) at position L96 a change to an amino acid selected from F and Y, particularly to Y; and/or
      (ac) at position L100 a change to amino acid T; and/or
(iii) making or causing in a parental VH3 antibody variable domain one or more of the following changes:
   (a) in the CDR regions:
      (aa) at position H50 a change to an amino acid selected from Q and T, particularly to T;
      (ab) at position H60 a change to amino acid N;
      (ac) at position H63 a change to an amino acid selected from V, I, and F;
      (ad) at position H64 a change to amino acid L;
      (ae) at position H95 a change to amino acid selected from D, N and T, particularly to D;
      (af) at position H102 a change to an amino acid selected from I and V, and/or
   (b) in the framework regions:
      (bb) at position H2 a change to amino acid A;
      (bc) at position H37 a change to amino acid I;
      (bd) at position H48 a change to amino acid I; and/or
      (be) at position H49 a change to amino acid G.

In a further aspect, the present invention relates to a method for modifying a parental antibody variable domain, comprising the step of:
(i) making or causing in a parental Vkappa1 antibody variable domain one or more of the following changes:
   (a) in the CDR regions:
      (ab) at position L94 a change to an amino acid selected from M, T, and V; and/or
      (ad) at position L32 a change to an amino acid selected from D, F, K, N, Q, S, and Y;
      (ae) at position L34 a change to an amino acid selected from A, S, and T, particularly to A or S, particularly to A;
      (af) at position L91 a change to an amino acid selected from A, G, S, and Y, particularly to Y; and/or
(ii) making or causing in a parental Vlambda1 antibody variable domain the following change:
   (a) in the CDR regions:
      (aa) at position L34 a change to amino acid G;
(iii) making or causing in a parental VH3 antibody variable domain one or more of the following changes:
   (a) in the CDR regions:
      (aa) at position H50 a change to amino acid S, particularly S, when said VH3 antibody variable domain is combined with a Vkappa antibody variable domain;
      (ag) at position H28 a change to amino acid P;
      (ah) at position H33 a change to amino acid A;
      (ai) at position H52 a change to an amino acid selected from D and S, particularly to D;
      (aj) at position H(103 minus 5) a change to amino acid G;
      (aj) one or two changes at positions H50 and H95 in order to create a salt bridge, particularly the following salt bridges: H50:R/H95:E; and H50:H/H95:E;
      (ak) one or two changes at positions H33 and H95 in order to create a salt bridge, particularly the following salt bridges: H33:R/H95:E; H33:R/H95:D; H33:H/H95:D; and H33:D/H95:H; and/or
   (b) in the framework regions:
      (ba) at position H2 a change to amino acid G.

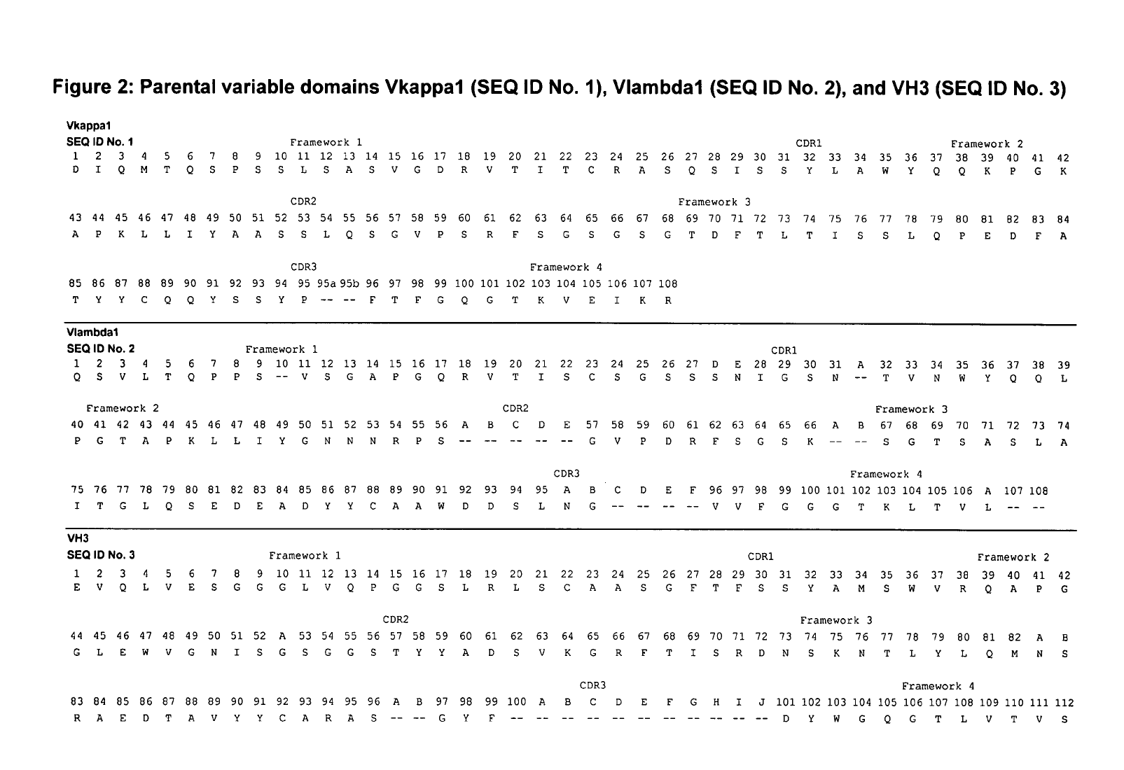

In the context of the present invention, the reference “H(103 minus 5)” refers to the fifth amino acid residue before the conserved residue H103:W. Such a nomenclature is necessary, since CDR3 of VH is of considerable length variability, so that the number, including any letter following a number (see FIG. 2, for example residues 96, 96A, 96B, 100, 100A etc.) that can be assigned for a residue with fixed distance from residue 103 will depend from the length of the CDR loop and is thus not clearly assignable.

In a further aspect, the present invention relates to a method for modifying a parental antibody variable domain, comprising the steps presented in sections [0061] and [0062]:

In the context of the present invention, the terms “Vlambda1”, and “VH3” refer to the subclasses of human antibody variable light (VL) and heavy (VH) chain domains as defined in WO 97/08320 (VH1a, VH1b, VH2, VH3, VH4, VH5, and VH6; Vkappa1, Vkappa2, Vkappa3 and Vkappa4; Vlambda1, Vlambda2 and Vlambda3). In this context, the term “subclass” refers to a group of variable domains sharing a high degree of identity and similarity, which can be represented by a consensus sequence for a given subclass. In the context of the present invention, the term “consensus sequence” refers to the HuCAL consensus genes as defined in WO 97/08320. The determination whether a given VL or VH domain belongs to a given VL or VH subclass is made by alignment of the respective variable domain with all known human germline segments (VBASE, Cook, G. P. & Tomlinson, I. M. (1995). The human immunoglobulin V-H repertoire. Immunology Today 16, 237-242) and determination of the highest degree of homology using a homology search matrix such as BLOSUM (Henikoff, S. & Henikoff, J. G. (1992). Amino acid substitution matrices from protein blocks. Proc. Natl. Acad. Sci. USA 89, 10915-10919). Methods for determining homologies and grouping of sequences according to homologies are well known to one of ordinary skill in the art. The grouping of the individual germline sequences into subclasses is done according to WO 97/08320.

In particular embodiments, said parental antibody variable domain is modified by making or causing at least one of the changes listed in (i)(a), (ii)(a) and (iii)(a).

In particular embodiments, the invention relates to a method, wherein at least two of said changes are made or caused, particularly wherein at least three of said changes are made or caused.

In certain embodiments, no change is made or caused at position L55. In certain other embodiments, no change is made or caused at position H95.

In a third aspect, the present invention relates to an antibody variable domain comprising at least one VL or VH domain selected from the group of:
(i) a Vkappa1 antibody variable domain based on the antibody variable domain of SEQ ID No. 1, comprising one or more of the following changes:
   (A) a single amino acid exchange L2:I to L2:T; or
   (B) at least two amino acid changes independently selected from the following group:
   (a) in the CDR regions:
      (aa) L55:Q to L55:Y;
      (ab) L94:T to L94:L; and/or
      (ac) L96:L to L96:Y; and/or
   (b) in the framework regions:
      (ba) L1:D to L1:A;
      (ba) L2:I to L2:T; and/or
      (bc) L70:D to L70:E;
   and optionally comprising up to 3 additional changes in the framework regions FR1 to FR3 different from those of (i)(A) and/or (B); provided that the antibody variable domains having the following accession numbers are excluded: AJ704539, U43767, 4762, 40096, 21224, CS483741, CS483744, U86790, X72459, 4753, 19244, AY043163, L26891, DQ184511, AY686924, 4806, DQ535161, 1S78_C, 1S78_E, and 1L7I_L (accession numbers according to Abysis (http://www.bioinf.org.uk/abysis/index.html); see Table 2 after Examples);
(ii) a Vlambda1 antibody variable domain based on the antibody variable domain of SEQ ID No. 2, comprising the following combination of changes:
   (a) in the CDR regions:
      (aa) L34:N to L34:S; and
      (ab) L96:V to L96:Y or L96:V to L96:F;
   and optionally further comprising up to 3 additional changes in the framework regions FR1 to FR3 different from those of (ii)(a);
(iii) a VH3 antibody variable domain based on the antibody variable domain of SEQ ID No. 3, comprising one or more of the following changes:
   (A) a single amino acid exchange selected from the following group:
   (a) in the CDR regions:
      (aa) H50V: to H50:T;
      (ab) H60A: to H60:N;
      (ac) H63V: to H63:I
      (ad) H63V: to H63:F; and
      (ae) H64:K to H64:L, provided that H:63 is not D;
   (B) at least two amino acid changes independently selected from the following group:
   (a) in the CDR regions:
      (aa) H50V: to H50:Q;
      (ab) H50V: to H50:T;
      (ac) H60A: to H60:N;
      (ad) H63V: to H63:I
      (ae) H63V: to H63:F;
      (af) H64:K to H64:L, provided that H:63 is not D; and
      (ag) H95:D to H95: N; and/or
   (b) in the framework regions:
      (ba) H2:V to H2:A;
      (bb) H37:V to H37:I;
      (bc) H48:V to H48:I; and/or
      (bd) H49:S to H49:G;
   in both (A) and (B) provided that the antibody variable domains having the following accession numbers are excluded: AM082547, AM082383, AM080583, AF471288, and AM082399 (accession numbers according to Abysis (http://www.bioinf.org.uk/abysis/index.html); see Table 2 after Examples).

In certain embodiments, the antibody variable domain comprises a VH and/of VL domain comprising at least three amino acid changes independently selected from the groups listed in (i)(B), (ii)(B) and (iii)(B).

In a fourth aspect, the present invention relates to a method for modifying an antibody variable domain, comprising the step of:
(i) making or causing in a Vkappa1 antibody variable domain having a sequence identity in the framework regions FR1 to FR3 of at least 90% to the antibody variable domain of SEQ ID No. 1 one or more of the following changes:
   (a) in the CDR regions:
      (aa) L55:Q to L55:(Y,H,W), particularly L55:Y;
      (ab) L94:T to L94:(F, H, I, K, L, R, Y), particularly L94:L; and/or
      (ac) L96:L to L96:(F,Y); and/or
   (b) in the framework regions:
      (ba) L1:D to L1:(A,D), particularly L1:A;
      (bb) L2:I to L2:T; and/or
      (bc) L70:D to L70:E;
(ii) making or causing in a Vlambda1 antibody variable domain having a sequence identity in the framework regions FR1 to FR3 of at least 90% to the antibody variable domain of SEQ ID No. 2 one or more of the following changes:
   (a) in the CDR regions:
      (aa) L34:N to L34:S;
      (ab) L96:V to L96:Y; and/or
      (ac) L96:V to L96:F; and/or
(iii) making or causing in a VH3 antibody variable domain having a sequence identity in the framework regions FR1 to FR3 of at least 90% to the antibody variable domain of SEQ ID No. 3 one or more of the following changes:
   (a) in the CDR regions:
      (aa) H50V: to H50:Q;
      (ab) H50V: to H50:T;
      (ac) H60A: to H60:V;
      (ad) H63V: to H63:I
      (ae) H63V: to H63:F
      (af) H63V: to H63:Q and/or
      (ag) H64:K to H64:L, provided that H:63 is not D; and
      (ah) H95:D to H95: N; and/or
   (b) in the framework regions:
      (ba) H2:V to H2:A;
      (bb) H37:V to H37:I;
      (bc) H48:V to H48:I; and/or
      (bd) H49:S to H49:G.

In an additional aspect, the present invention relates to a method for modifying an antibody variable domain, comprising the step of:
(i) making or causing in a Vkappa1 antibody variable domain having a sequence identity in the framework regions FR1 to FR3 of at least 90% to the antibody variable domain of SEQ ID No. 1 one or more of the following changes:
   (a) in the CDR regions:
      (ab) L94:T to L94:(M, T, V); and/or
      (ad) L32:Y to L32(D, F, K, N, Q, S); and/or
(iii) making or causing in a VH3 antibody variable domain having a sequence identity in the framework regions FR1 to FR3 of at least 90% to the antibody variable domain of SEQ ID No. 3 one or more of the following changes:
   (a) in the CDR regions:
      (ai) H50:V to H50:S, particularly when said VH3 antibody variable domain is combined with a Vkappa antibody variable domain;
      (aj) H28:T to H28:P;
      (ak) H52:S to H52:D;
      (al) H(103-5):X to H(103-5):G;
      (am) H50/H95 to a salt bridge, particularly a salt bridge selected from: H50:R/H95:E; and H50:H/H95:E; and/or
      (an) H33/H95 to a salt bridge, particularly a salt bridge selected from: H33:R/H95:E; H33:R/H95:D; H33:H/H95:D; and H33:D/H95:H.

In certain embodiments, no change is made or caused at position L55. In certain other embodiments, no change is made or caused at position H95.

In certain embodiments, the method comprises to make or cause at least three amino acid changes independently selected from the groups listed in (i), (ii) and (iii).

In a fifth aspect, the present invention relates to the use of an antibody variable domain according to the present invention, or an antibody variable domain modified according to the present invention, in the construction of a diverse collection of antibody variable domains, comprising the step of:
(a) diversifying one or more amino acid positions in one or more CDR regions of said antibody variable domain, provided that
   (aa) none of the following CDR positions is diversified: Vkappa1: L96; Vlambda1: L96; VH3: H50 and H95; and the following CDR positions are each independently optionally diversified: Vkappa1: L55 and L94; VH3: H60, H63, and H64; or
   (ab) any of the following CDR positions is not diversified, if it carries one of the following amino acid residues: Vkappa1: L55:Y, L94:L, L96:Y; Vlambda1: L96:Y; VH3: H50:T, H60:N, H63:I, H64:L, and H95:D; or
   (ac) any of the following CDR positions is either not diversified, or it is diversified with a bias towards the following amino acid residues: Vkappa1: L55:Y, L94:L, L96:Y; Vlambda1: L96:Y; VH3: H50:T, H60:N, H63:I, H64:L, and H95:D; particularly wherein the listed amino acid residues is present to at least 30%, and more particularly to at least 50% in the diversification mixture; or
   (ad) any of the following CDR positions is either not diversified or diversified with the indicated limited diversity only: Vkappa1: L55:YHW, L94:FHIKLRY, L96:FY; Vlambda1: L96:FY; VH3: H50:QT, H60:HNRS, H63:VIF, H64:KL, and H95:DNT.

In particular embodiments, none of the following CDR positions is diversified: Vkappa1: L55, L94, L96; Vlambda1: L96; VH3: H50, H60, H63, H64, and H95.

In particular additional embodiments, the following diversification schemes are used for Vkappa libraries:
(aa) CDR residue L34 is fixed to A, when CDR1 of Vkappa is diversified;
(ab) CDR residue L34:A is diversified with limited variability comprising only A, S, and T, or only A and S;
(ac) CDR residue L91 is fixed to Y, when CDR3 of Vkappa is diversified;
(ad) CDR residue L94 is fixed to L, when CDR3 of Vkappa is diversified;
(ae) CDR residue L94:L is diversified with limited variability comprising only residues selected from YFILMVHRK;
(af) CDR residue L96 is fixed to Y, when CDR3 of Vkappa is diversified;
(ag) CDR residues L96:Y and L104:L are not varied, when CDR3 of Vkappa is diversified;
(ah) CDR residue L96 is fixed to I, when CDR3 of Vkappa is diversified;
(ai) CDR residue L96 is diversified with limited variability comprising only residues selected from Y and I;

In particular additional embodiments, the following diversification schemes are used for Vlambda libraries:
(ba) CDR residue L34 is fixed to S, when CDR1 of Vlambda is diversified;
(bb) CDR residue L96 is fixed to Y, when CDR3 of Vlambda is diversified;
(bc) CDR residues L96:Y, L100:T, and L104:V are not varied, when CDR3 of Vlambda is diversified;
(bd) CDR residue L96 is diversified with limited variability comprising only residues selected from Y and F.

In particular additional embodiments, the following diversification schemes are used for VH libraries:
(ca) CDR residues H(103-5) is fixed to G, residue H(103-3) is fixed to F, and diversity is limited to FY at residue H(103-4), when CDR3 of VH is diversified;
(cb) CDR residues H(103-5) is fixed to G, residue H(103-3) is fixed to M, and diversity is limited to FY at residue H(103-4), when CDR3 of VH is diversified;
(cc) CDR residues 95 is fixed to S or T, if the CDR3 length is 8 (according to Kabat), when CDR3 of VH is diversified;
(cd) CDR residues 95 is fixed to D, if the CDR3 length is 7 (according to Kabat), when CDR3 of VH is diversified;
(ce) CDR residues 60 is fixed to N, when CDR2 of VH is diversified, particularly when CDR2 of VH3 is diversified;
(cf) CDR residues 60 is biased towards N, when CDR2 of VH is diversified, particularly when CDR2 of VH3 is diversified;
(cg) a salt bridge between residues H50 and H95 is maintained in a library wherein CDR2 and/or CDR3 of VH is diversified, particularly H50:R/H95:E; H50:H/H95:E; H50:(R/H)/H95:(D/E);
(ch) CDR residue 28 is fixed to P, when CDR1 of VH is diversified, particularly in a VH3 library;
(ci) framework residue 2 is fixed to A or G, particularly in a VH3 library;
(cj) framework residues 48 and 49 are fixed to I, and G, respectively, and CDR residue 50 is fixed to Q, when CDR2 of VH is diversified;
(ck) framework residue 49 is fixed to G, and CDR residue 50 is fixed to S or T, when CDR2 of VH is diversified, particularly if H32 is N and/or H33 is Y; and/or
(cl) framework residue 49 is fixed to G, and CDR residue 50 is fixed to Q, when CDR2 of VH is diversified, if H32 is Y and/or H33 is A.

In a sixth aspect, the present invention relates to a method for construction of a diverse collection of antibody variable domains, comprising the step of (a) diversifying one or more amino acid positions in one or more CDR regions of an antibody variable domain according to claim 1, or an antibody variable domain modified according to the method of claim 2, provided that
(aa) none of the following CDR positions is diversified: Vkappa1: L96; Vlambda1: L96; VH3: H50 and H95; and the following CDR positions are each independently optionally diversified: Vkappa1: L55 and L94; VH3: H60, H63, and H64; or
(ab) any of the following CDR positions is not diversified, if it carries one of the following amino acid residues: Vkappa1: L55:Y, L94:L, L96:Y; Vlambda1: L96:Y; VH3: H50:T, H60:N, H63:I, H64:L, and H95:D; or
(ac) any of the following CDR positions is either not diversified, or it is diversified with a bias towards the following amino acid residues: Vkappa1: L55:Y, L94:L, L96:Y; Vlambda1: L96:Y; VH3: H50:T, H60:N, H63:I, H64:L, and H95:D; particularly wherein the listed amino acid residues is present to at least 30%, and more particularly to at least 50% in the diversification mixture; or
(ad) any of the following CDR positions is either not diversified or diversified with the indicated limited diversity only: Vkappa1: L55:YHW, L94:FHIKLRY, L96:FY; Vlambda1: L96:FY; VH3: H50:QT, H60:HNRS, H63:VIF, H64:KL, and H95:DNT.

In particular embodiments, none of the following CDR positions is diversified: Vkappa1: L55, L94, L96; Vlambda1: L96; VH3: H50, H60, H63, H64, and H95.

In a seventh aspect, the present invention relates to a diverse collection of antibody variable domains, wherein said collection comprises one or more diverse collections of amino acid residues at one or more positions in one or more CDR regions, provided that
(aa) none of the following CDR positions is diversified: Vkappa1: L96; Vlambda1: L96; VH3: H50 and H95; and the following CDR positions are each independently optionally diversified: Vkappa1: L55 and L94; VH3: H60, H63, and H64; or
(ab) any of the following CDR positions is not diversified, if it carries one of the following amino acid residues: Vkappa1: L55:Y, L94:L, L96:Y; Vlambda1: L96:Y; VH3: H50:T, H60:N, H63:I, H64:L, and H95:D; or
(ac) any of the following CDR positions is either not diversified, or it is diversified with a bias towards the following amino acid residues: Vkappa1: L55:Y, L94:L, L96:Y; Vlambda1: L96:Y; VH3: H50:T, H60:N, H63:I, H64:L, and H95:D; particularly wherein the listed amino acid residues is present to at least 30%, and more particularly to at least 50% in the diversification mixture; or
(ad) any of the following CDR positions is either not diversified or diversified with the indicated limited diversity only: Vkappa1: L55:YHW, L94:FHIKLRY, L96:FY; Vlambda1: L96:FY; VH3: H50:QT, H60:HNRS, H63:VIF, H64:KL, and H95:DNT;
wherein the antibody variable domain is selected from the group of:
(i) a Vkappa1 antibody variable domain having a sequence identity in the framework regions FR1 to FR3 of at least 90% to the antibody variable domain of SEQ ID No. 1, optionally comprising one or more of the following changes:
   (b) in the framework regions:
      (ba) L1:D to L1:A;
      (bb) L2:I to L2:T; and/or
      (bc) L70:D to L70:E;
(ii) a Vlambda1 antibody variable domain having a sequence identity in the framework regions FR1 to FR3 of at least 90% to the antibody variable domain of SEQ ID No. 2; and/or
(iii) a VH3 antibody variable domain having a sequence identity in the framework regions FR1 to FR3 of at least 90% to the antibody variable domain of SEQ ID No. 3, optionally comprising one or more of the following changes:
   (b) in the framework regions:
      (ba) H2:V to H2:A;
      (bb) H37:V to H37:I;
      (bc) H48:V to H48:I; and/or
      (bd) H49:S to H49:G.

In particular embodiments, none of the following CDR positions is diversified: Vkappa1: L55, L94, L96; Vlambda1: L96; VH3: H50, H60, H63, H64, and H95.

In an eighth aspect, the present invention relates to an antibody that has a melting temperature of the Fab fragment of significantly above 92° C. when analyzed by differential scanning calorimetry in pure 1× phosphate buffered saline pH 7.4, (containing 1.06 mM KH₂PO₄, 2.97 mM Na₂HPO₄×7 H₂O, 155.17 mM NaCl and no other supplements) using a scan-rate of 60° C. per hour, no gain and a scan range of 32° C. to 115° C.

In one embodiment, the melting temperature of the Fab fragment is above 100° C.

In a ninth aspect, the present invention relates to a nucleic acid sequence encoding the antibody or functional fragment thereof according to the present invention.

In a tenth aspect, the present invention relates to a vector comprising the nucleic acid sequence according to the present invention.

In an eleventh aspect, the present invention relates to a host cell comprising the nucleic acid sequence according to the present invention, or the vector according to the present invention.

In a twelfth aspect, the present invention relates to a method for generating the antibody or functional fragment thereof according to the present invention, comprising the step of expressing the nucleic acid sequence according to the present invention, or the vector according to the present invention, either in vitro or in an appropriate host cell, including the host cell according to the present invention.

In a thirteenth aspect, the present invention relates to pharmaceutical compositions comprising an antibody molecule or functional fragment thereof, and optionally a pharmaceutically acceptable carrier and/or excipient. The compositions may be formulated e.g. for once-a-day administration, twice-a-day administration, or three times a day administration.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). The term “pharmaceutically acceptable” may also mean approved by a regulatory agency of the Federal or a state government or listed in the U.S. Pharmacopeia or other generally recognized pharmacopeia for use in mammals, and more particularly in humans.

In the context of the present invention, the term “about” or “approximately” means between 90% and 110% of a given value or range.

The term “carrier” applied to pharmaceutical compositions of the invention refers to a diluent, excipient, or vehicle with which an active compound (e.g., a bispecific antibody fragment) is administered. Such pharmaceutical carriers may be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by A. R. Gennaro, 20th Edition.

The active ingredient (e.g., a modified antibody fragment) or the composition of the present invention may be used for the treatment of at least one disease or disorders, wherein the treatment is adapted to or appropriately prepared for a specific administration as disclosed herein (e.g., to once-a-day, twice-a-day, or three times a day administration). For this purpose the package leaflet and/or the patient information contains corresponding information.

The active ingredient (e.g., the modified antibody molecule or fragment thereof) or the composition of the present invention may be used for the manufacture of a medicament for the treatment of at least one disease or disorder, wherein the medicament is adapted to or appropriately prepared for a specific administration as disclosed herein (e.g., to once-a-day, twice-a-day, or three times a day administration). For this purpose the package leaflet and/or the patient information contains corresponding information.

### EXAMPLES

The following examples illustrate the invention without limiting its scope.

### Example 1

### Antibody Cloning

Antibody genes were designed based on the desired amino acid sequence and purchased as synthetic genes or synthetic gene fragments from GeneArt or DNA2.0. Genes encoding antibody variants with point mutations were generated by PCR or overlap PCR, using the polymerase Pwo Master, purchased from Roche, and synthetic oligonucleotides encoding the desired point mutations, purchased from Thermo Fisher Scientific, according to manufacturer's instructions. An E. coli Fab expression vector was generated by modification of the plasmid pUC19, which was purchased from New England Biolabs. The pUC19 backbone was modified by the addition of two synthetic ribosome binding sites driving expression of antibody heavy and light chains, two synthetic signal peptide sequences driving the secretion of antibody chains into the E. coli periplasm and one M13 phage origin potentially enabling single strand production. Synthetic antibody genes, synthetic fragments of antibody genes and PCR-generated variants of antibody genes encoding point mutations were cloned into this E. coli Fab expression vector by restriction digestion, using restriction endonucleases purchased from Roche, followed by ligation, using LigaFast purchased from Promega, according to manufacturer's instructions. Ligation reactions were transformed into competent TG1 E. coli cells purchased from Stratagene or Zymoresearch.

### Example 2

### Antibody Expression and Purification

TG1 E. coli clones bearing Fab expression constructs were grown in LB and TB solid and liquid media, purchased from Carl Roth, which were supplemented with Carbenicillin and glucose, purchased from VWR. Antibody expression in liquid cultures was performed overnight in Erlenmeyer flasks in a shaking incubator and was induced by the addition of isopropyl-β-D-thiogalactopyranoside (IPTG), purchased from Carl Roth, to the growth medium. Culture supernatants containing secreted Fab fragments were clarified by centrifugation of the expression cultures. Clarified culture supernatants were supplemented with a 1% volume of Streptomycin/Penicillin solution, purchased from PAA Laboratories, a 2% volume of 1M Tris pH8.0, purchased from VWR, and a 0.4% volume of STREAMLINE rProtein A resin, purchased from GE Healthcare. The supplemented culture supernatants were incubated on a rolling incubator for 3 hours or overnight to achieve binding of Fab fragments to the protein A resin. Resins were then transferred into gravity flow columns, washed once using 30 bedvolumes of 2×PBS pH 7.4, purchased from Invitrogen, washed once using 5 bedvolumes of a buffer containing 10 mM Tris pH 6.8 and 100 mM NaCl, purchased from VWR, and eluted using a buffer containing 10 mM citric acid pH3 and 100 mM NaCl, purchased from VWR. Eluted Fab fragments were neutralized by adding an 8% volume of 1M Tris pH 8.0. Neutralized purified Fab fragments were buffer exchanged into pure 1×PBS pH 7.4 (containing 1.06 mM KH₂PO₄, 2.97 mM Na₂HPO₄×7H2O, 155.17 mM NaCl and no other supplements; Invitrogen catalogue No. 10010056), using illustra NAP-5 desalting columns from GE Healthcare, according to manufacturer's instructions.

### Example 3

### Antibody Stability Measurement

The biophysical stability of purified, buffer-exchanged Fab fragments was determined in 1×PBS pH 7.4 (Invitrogen catalogue No. 10010056) using differential scanning calorimetry (DSC). For all measurements, a capillary cell microcalorimeter equipped with autosampler and controlled by VPViewer2000 CapDSC software from MicroCal was used. All Fab fragments were scanned against pure buffer containing no antibody (1×PBS pH 7.4; Invitrogen catalogue No. 10010056). The scan parameters were set to analyse a temperature window from 32° C. to between 105° C. and 115° C., with a pre-scan thermostat of 2 minutes, a post-scan thermostat of 0 minutes and no gain. The scan rate was set to 250° C. per hour for screening applications and to 60° C. per hour for re-analysis of the most stable combination mutants. The absolute melting temperature of the Fab fragments determined in screening mode (scan-rate 250° C. per hour) was 3.7° C. to 4.5° C. higher than in re-analysis mode (scan-rate 60° C. per hour), but ranking of clones was the same in both modes. Melting temperatures of Fab fragments were determined after PBS reference subtraction, using Origin 7.0 software from MicroCal.

The following Table 1 shows a compilation of experimental data obtained with mutants of Vkappa1/VH3 and Vlambda1/VH3 Fab fragments.

TABLE 1
Construction and testing of mutants.

| | | TM in 1x |
|---|---|---|
| | | PBS pH7.4 |
| | | with scan- |
| Example | | rate |
| No. | Starting Sequence and Position/Residue tested | 250° C./hour |
| Example 1 | | |
| VH | EVQLVESGGGLVQPGGSLRLSCAASGFTFSSYAMSWVRQAPGKGLEWVGNISGSG | |
| | GSTYYADSVKGRFTISRDNSKNTLYLQMNSLRAEDTAVYYCARDSGYFDYWGQGTL | |
| | VTVSS | |
| VL | DTQMTQSPSSLSASVGDRVTITCRASQSISSYLAWYQQKPGKAPKLLIYAASSLQSGV | |
| | PSRFSGSGSGTDFTLTISSLQPEDFATYYCQQYSSYPFTFGQGTKVEIKR | |
| | VK F96 | 93.3 |
| | VK W96 | 93.5 |
| | VK Y96 | 95.5 |
| | VK H96 | 95.3 |
| | VK M96 | 91.2 |
| | VK L96 | 92.8 |
| | VK I96 | 95.3 |
| | VK delta 96 | 94.6 |
| Example 2 | | |
| VH | EVQLVESGGGLVQPGGSLRLSCAASGFTFSSYAMSWVRQAPGKGLEWVGNISGSGG | |
| | ST | |
| | YYADSVKGRFTISRDNSKNTLYLQMNSLRAEDTAVYYCARDSGYFDYWGQGTLVTVS | |
| | S | |
| VL | DTQMTQSPSSLSASVGDRVTITCRASQSISSYLNWYQQKPGKAPKLLIYAASSLQSGVP | |
| | SR | |
| | FSGSGSGTDFTLTISSLQPEDFATYYCQQYSSYPFTFGQGTKVEIKR | |
| | VK F96 | 95.5 |
| | VK W96 | 94.3 |
| | VK Y96 | 95.9 |
| | VK H96 | 93.2 |
| | VK M96 | 94.8 |
| | VK L96 | 95 |
| | VK I96 | 95.6 |
| | VK delta 96 | N.D. |
| Example 3 | | |
| VH | EVQLVESGGGLVQPGGSLRLSCAASGFTFSSYAMSWVRQAPGKGLEWVGNISGSGG | |
| | ST | |
| | YYADSVKGRFTISRDNSKNTLYLQMNSLRAEDTAVYYCARDSGYMDYWGQGTLVTV | |
| | SS | |
| VL | DTQMTQSPSSLSASVGDRVTITCRASQSISSYLAWYQQKPGKAPKLLIYAASSLQSGVP | |
| | SR | |
| | FSGSGSGTDFTLTISSLQPEDFATYYCQQYSSYPFTFGQGTKVEIKR | |
| 2.19 | VK F96 | 91.8 |
| 2.17 | VK W96 | 94.3 |
| 2.18 | VK Y96 | 95.4 |
| 2.20 | VK H96 | N.D. |
| 2.21 | VK M96 | 88 |
| 2.22 | VK L96 | 92 |
| 2.23 | VK I96 | 93.3 |
| 2.24 | VK delta 96 | 94.5 |
| Example 4 | | |
| VH | EVQLVESGGGLVQPGGSLRLSCAASGFTFSSYAMSVVVRQAPGKGLEWVGNISGSGG | |
| | ST | |
| | YVADSVKGRFTISRDNSKNTLYLQMNSLRAEDTAVYYCARDSGYMDYWGQGTLVTV | |
| | SS | |
| VL | DTQMTQSPSSLSASVGDRVTITCRASQSISSYLNWYQQKPGKAPKLLIYAASSLQSGVP | |
| | S | |
| | RFSGSGSGTDFTLTISSLQPEDFATYYCQQYSSYPFTFGQGTKVEIKR | |
| | VK F96 | 95 |
| | VK W96 | N.D. |
| | VK Y96 | 95.9 |
| | VK H96 | 93.8 |
| | VK M96 | 94.2 |
| | VK L96 | 94.8 |
| | VK I96 | 95.2 |
| | VK delta 96 | 89.3 |
| Example 5 | | |
| VH | EVQLVESGGGLVQPGGSLRLSCAASGFTFSSYAMSWVRQAPGKGLEWVGNISGSGG | |
| | ST | |
| | YYADSVKGRFTISRDNSKNTLYLQMNSLRAEDTAVYYCARDSGYFDYWGQGTLVTVS | |
| | SS | |
| VL | DIQMTQSPSSLSASVGDRVTITCRASQSISSYLAWYQQKPGKAPKLLIYAASSLQSGVP | |
| | S | |
| | RFSGSGSGTDFTLTISSLQPEDFATYYCQQYSSYPFTFGQGTKVEIKR | |
| | VH Y102 | 94.7 |
| | VH H102 | 94.1 |
| | VH I102 | 95.2 |
| | VH V102 | 95.2 |
| Example 6 | | |
| VH | ETQLVESGGGLVQPGGSLRLSCAASGFTFSSYAMSWVRQAPGKGLEWVGNISGSGG | |
| | STYY | |
| | ADSVKGRFTISRDNSKNTLYLQMNSLRAEDTAVYYCARDSGYFDYWGQGTLVTVSS | |
| VL | DIQMTQSPSSLSASVGDRVTITCRASQSISSYLAWYQQKPGKAPKLLIYAASSLQSGVP | |
| | SRFS | |
| | GSGSGTDFTLTISSLQPEDFATYYCQQYSSYPFTFGQGTKVEIKR | |
| | VH Y102 | 89.3 |
| | VH H102 | 89.3 |
| | VH I102 | 92.3 |
| | VH V102 | 92.5 |
| Example 7 | | |
| VH | EVQLVESGGGLVQPGGSLRLSCAASGFTFSSNYMSWVRQAPGKGLEWVGNISGSGG | |
| | STY | |
| | YADSVKGRFTISRDNSKNTLYLQMNSLRAEDTAVYYCARDSGYFDIWGQGTLVTVSS | |
| VL | DTQMTQSPSSLSASVGDRVTITCRASQSISSYLAVVYQQKPGKAPKWYAASSLQSGVP | |
| | SRF | |
| | SGSGSGTDFTLTISSLQPEDFATYYCQQYSSYPYTFGQGTKVEIKR | |
| | VH N50 | 90 |
| | VH Y50 | 81 |
| | VH H50 | 88 |
| | VH Q50 | 87 |
| | VH R50 | 86 |
| | VH T50 | 95.3 |
| Example 8 | | |
| VH | EVQLVESGGGLVQPGGSLRLSCAASGFTFSSYAMSWVRQAPGKGLEWVGNISGSGG | |
| | STY | |
| | YADSVKGRFTISRDNSKNTLYLQMNSLRAEDTAVYYCARDSGYFDIWGQGTLVTVSS | |
| VL | DTQMTQSPSSLSASVGDRVTITCRASQSISSYLAWYQQKPGKAPKLLIYAASSLQSGVP | |
| | SRF | |
| | SGSGSGTDFTLTISSLQPEDFATYYCQQYSSYPYTFGQGTKVEIKR | |
| | VH N50 | 95.5 |
| | VH Y50 | 95 |
| | VH H50 | 95 |
| | VH Q50 | 95.4 |
| | VH R50 | 95 |
| | VH T50 | 95.9 |
| Example 9 | | |
| VH | EVQLVESGGGLVQPGGSLRLSCAASGFTFSSYAMSWVRQAPGKGLEWVGNISGSGG | |
| | S | |
| | TYYADSVKG RFTISRDNSKNTLYLQM NSLRAEDTAVYYCARDSGYFDIWGQGTLVTVS | |
| | S | |
| VL | DTQMTQSPSSLSASVGDRVTITCRASQSISSYLAWYQQKPGKAPKLLIYAASSLQSGVP | |
| | S | |
| | RFSGSGSGTDFTLTISSLQPEDFATYYCQQYSSYPYTFGQGTKVEIKR | |
| | VH A60 | 95.5 |
| | VH N60 | 96.2 |
| | VH S60 | 95.5 |
| | VH P60 | 92.9 |
| Example 10 | | |
| VH | EVQLVESGGGLVQPGGSLRLSCAASGFTFSSYAMSWIRQAPGKGLEWIGQISGSGGS | |
| | TYY | |
| | NDSVKGRFTISRDNSKNTLYLQMNSLRAEDTAVYYCARDSGYFDIWGQGTLVTVSS | |
| VL | DIQMTQSPSSLSASVGDRVTITCRASQSISSYLAWYQQKPGKAPKLLIYAASSLQSGVP | |
| | SRF | |
| | SGSGSGTDFTLTISSLQPEDFATYYCQQYSSYPYTFGQGTKVEIKR | |
| | VH N60 | 98.2 |
| | VH H60 | 96.9 |
| | VH T60 | 95.9 |
| Example 11 | | |
| VH | EVQLVESGGGLVQPGGSLRLSCAASGFTFSSYAMSWVRQAPGKGLEWVGNISGSGG | |
| | ST | |
| | YYADSVKGRFTISRDNSKNTLYLQMNSLRAEDTAVYYCARDSGYFDIWGQGTLVTVSS | |
| VL | DTQMTQSPSSLSASVGDRVTITCRASQSISSYLAWYQQKPGKAPKLLIYAASSLQSGVP | |
| | SR | |
| | FSGSGSGTDFTLTISSLQPEDFATYYCQQYSSYPYTFGQGTKVEIKR | |
| | VH G50 | 95.5 |
| | VH A50 | 95 |
| | VH S50 | 94.6 |
| Example 12 | | |
| VH | EVQLVESGGGLVQPGGSLRLSCAASGFTFSSYAMSWVRQAPGKGLEWVGQISGSGG | |
| | STY | |
| | YNDSVKGRFTISRDNSKNTLYLQMNSLRAEDTAVYYCARDSGYFD1WGQGTLVTVSS | |
| VL | DTQMTQSPSSLSASVGDRVTITCRASQSISSYLAWYQQKPGKAPKWYAASSLQSGVP | |
| | SRF | |
| | SGSGSGTDFTLTISSLQPEDFATYYCQQYSSYPYTFGQGTKVEIKR | |
| | VH V48 | 97.4 |
| | VH 148 | 98 |
| | VH M48 | 94.8 |
| Example 13 | | |
| VH | EVQLVESGGGLVQPGGSLRLSCAASGFTFSSYAMSWIRQAPGKGLEWVGQISGSGGS | |
| | TYY | |
| | NDSVKGRFTISRDNSKNTLYLQMNSLRAEDTAVYYCARDSGYFDIWGQGTLVTVSS | |
| VL | DTQMTQSPSSLSASVGDRVTITCRASQSISSYLAWYQQKPGKAPKLLIYAASSLQSGVP | |
| | SRF | |
| | SGSGSGTDFTLTISSLQPEDFATYYCQQYSSYPYTFGQGTKVEIKR | |
| | VH V48 | 97.7 |
| | VH I48 | 98.1 |
| | VH M48 | 95.2 |
| Example 14 | | |
| VH | EVQLVESGGGLVQPGGSLRLSCAASGFTFSSYAMSWVRQAPGKGLEWVGQISGSGG | |
| | STYY | |
| | NDSVKGRFTISRDNSKNTLYLQMNSLRAEDTAVYYCARDSGYFDIWGQGTLVTVSS | |
| VL | DTQMTQSPSSLSASVGDRVTITCRASQSISSYLAWYQQKPGKAPKLLIYAASSLQSGVP | |
| | SRFS | |
| | GSGSGTDFTLTISSLQPEDFATYYCQQYSSYPYTFGQGTKVEIKR | |
| | VH V37 | 97.4 |
| | VH 137 | 97.7 |
| Example 15 | | |
| VH | EVQLVESGGGLVQPGGSLRLSCAASGFTFSSYAMSWVRQAPGKGLEWIGQISGSGGS | |
| | TYYN | |
| | DSVKGRFTISRDNSKNTLYLQMNSLRAEDTAVYYCARDSGYFDIWGQGTLVTVSS | |
| VL | DTQMTQSPSSLSASVGDRVTITCRASQSISSYLAWYQQKPGKAPKLLIYAASSLQSGVP | |
| | SRFS | |
| | GSGSGTDFTLTISSLQPEDFATYYCQQYSSYPYTFGQGTKVEIKR | |
| | VH V37 | 98 |
| | VH I37 | 98.1 |
| Example 16 | | |
| VH | EVQLVESGGGLVQPGGSLRLSCAASGFTFSSYAMSWVRQAPGKGLEWMGQISGSG | |
| | GSTYY | |
| | NDSVKGRFTISRDNSKNTLYLQMNSLRAEDTAVYYCARDSGYFDIWGQGTLVTVSS | |
| VL | DTQMTQSPSSLSASVGDRVTITCRASQSISSYLAWYQQKPGKAPKLLIYAASSLQSGVP | |
| | SRFSG | |
| | SGSGTDFTLTISSLQPEDFATYYCQQYSSYPYTFGQGTKVEIKR | |
| | VH V37 | 94.8 |
| | VH 137 | 95.2 |
| Example 17 | | |
| VH | EVQLVESGGGLVQPGGSLRLSCAASGFTFSSYAMSWIRQAPGKGLEWIGQISGSGGS | |
| | TYYND | |
| | SVKGRFTISRDNSKNTLYLQMNSLRAEDTAVYYCARDSGYFDIWGQGTLVTVSS | |
| VL | DTQMTQSPSSLSASVGDRVTITCRASQSISSYLAWYQQKPGKAPKLLIYAASSLQSGVP | |
| | SRFSG | |
| | SGSGTDFTLTISSLQPEDFATYYCQQYSSYPYTFGQGTKVEIKR | |
| | VK D1 | 97 |
| | VK W1 | 97.5 |
| | VK Y1 | 98.4 |
| | VK R1 | 98.2 |
| | VK A1 | 98.2 |
| Example 18 | | |
| VH | EVQLVESGGGLVQPGGSLRLSCAASGFTFSSYAMSWIRQAPGKGLEWIGQISGSGGS | |
| | TYYNDS | |
| | VKGRFTISRDNSKNTLYLQMNSLRAEDTAVYYCARDSGYFDIWGQGTLVTVSS | |
| VL | DTQMTQSPSSLSASVGDRVTITCRASQSISSYLAWYQQKPGKAPKLLIYAASSLQSGVP | |
| | SRFSGS | |
| | GSGTDFTLTISSLQPEDFATYYCQQYSSYPYTFGQGTKVEIKR | |
| | VK Q55 | 97 |
| | VK W55 | 98.1 |
| | VK Y55 | 98.7 |
| | VK H55 | 98.1 |
| | VK R55 | 96.2 |
| Example 19 | | |
| VH | EVQLVESGGGLVQPGGSLRLSCAASGFTFSSYAMSWIRQAPGKGLEWIGQISGSGGS | |
| | TYYNDSV | |
| | KGRFTISRDNSKNTLYLQMNSLRAEDTAVYYCARDSGYFDIWGQGTLVTVSS | |
| VL | DTQMTQSPSSLSASVGDRVTITCRASQSISSYLAWYQQKPGKAPKLLIYAASSLQSGVP | |
| | SRFSGSGS | |
| | GTDFTLTISSLQPEDFATYYCQQYSSYPYTFGQGTKVEIKR | |
| | VK D70 | 97 |
| | VK E70 | 97.5 |
| | EVQLVESGGGLVQPGGSLRLSCAASGFTFSSYAMSWIRQAPGKGLEWIGQISGSGGS | |
| | TYYNDSV | |
| | KGRFTISRDNSKNTLYLQMNSLRAEDTAVYYCARDSGYFDIWGQGTLVTVSS | |
| | DIQMTQSPSSLSASVGDRVTITCRASQSISSYLAWYQQKPGKAPKLLIYAASSLQSGVP | |
| | SRFSGSG | |
| | SGTDFTLTISSLOPEDFATYYCQQYSSYPYTFGQGTKVEIKR | |
| | VH V2 | 98.2 |
| | VH A2 | 99.2 |
| | VH N2 | 96.2 |
| | VH L2 | 97.8 |
| Example 20 | | |
| VH | EVQLVESGGGLVQPGGSLRLSCAASGFTFSSYAMSWIRQAPGKGLEWIGQISGSGGS | |
| | TYYNDS | |
| | VKGRFTISRDNSKNTLYLQMNSLRAEDTAVYYCARDSGYFDIWGQGTLVTVSS | |
| VL | DTQMTQSPSSLSASVGDRVTITCRASQSISSYLAWYQQKPGKAPKLLIYAASSLYSGVP | |
| | SRFSGSG | |
| | SGTDFTLTISSLQPEDFATYYCQQYSSYPYTFGQGTKVEIKR | |
| | VK Y94 | 98.7 |
| | VK W94 | 95.7 |
| | VK H94 | 97 |
| | VK R94 | 98.4 |
| | VK L94 | 101 |
| | VK N94 | 94.6 |
| | VK S94 | 94.6 |
| | VK 194 | 99.9 |
| | VK F94 | 99 |
| | VK M94 | 99.9 |
| | VK V94 | 98.6 |
| Example 21 | | |
| VH | EVQLVESGGGLVQPGGSLRLSCAASGFTFSSYAMSWVRQAPGKGLEWVGTISGSGG | |
| | STYYAD | |
| | NVLGRFTISRDNSKNTLYLQM NSLRAEDTAVYYCARASGYFDYWGQGTLVTVSS | |
| VL | SSVLTQPPSVSGAPGQRVTISCSGSSSNIGSNIVNWYQQLPGTAPKWYGNNNRPSG | |
| | VPDRF | |
| | SGSKSGTSASLAITGLQSEDEADYYCAAWDDSLNGVVFGGGTKLTVL | |
| | VH A95 | 87.9 |
| | VH D95 | 91.5 |
| | VH H95 | 88.7 |
| | VH N95 | 89.8 |
| | VH S95 | 89.4 |
| | VH T95 | 89.7 |
| Example 22 | | |
| VH | EVQLVESGGGLVQPGGSLRLSCAASGFTFSSNYMSWVRQAPGKGLEWVGTISGSGG | |
| | STYYADN | |
| | VLGRFTISRDNSKNTLYLQMNSLRAEDTAVYYCARDSSGYFDYWGQGTLVTVSS | |
| VL | SSVLTQPPSVSGAPGQRVTISCSGSSSNIGSNIVNWYQQLPGTAPKWYGNNNRPSG | |
| | VPDRFSG | |
| | SKSGTSASLAITGLQSEDEADYYCAAWDDSLNGVVFGGGTKLTVL | |
| | VH D95 | 86.1 |
| | VH H95 | 85.7 |
| | VH N95 | 86.5 |
| | VH S95 | 86.6 |
| | VH T95 | 87.7 |
| Example 23 | | |
| VH | EVOLVESGGGLVQPGGSLRLSCAASGFTFSSYAMSWVRQAPGKGLEWVGTISGSGG | |
| | STYYA | |
| | DNVLGRFTISRDNSKNTLYLQMNSLRAEDTAVYYCARDSGYFDYWGQGTLVTVSS | |
| VL | SSVLTQPPSVSGAPGQRVTISCSGSSSNIGSNTVNWYQQLPGTAPKLLIYGNNNRPSG | |
| | VPDRF | |
| | SGSKSGTSASLAITGLQSEDEADYYCAAWDDSLNGVVFGGGTKLTVL | |
| | VL V96 | 91.7 |
| | VL W96 | 91.8 |
| | VL Y96 | 91.8 |
| | VL F96 | 89.6 |
| | VL H96 | 90.5 |
| | VL M96 | 90.5 |
| | VL L96 | 90.8 |
| | VL 196 | 91.2 |
| Example 24 | | |
| VH | EVQLVESGGGLVQPGGSLRLSCAASGFTFSSNYMSWVRQAPGKGLEWVGTISGSGG | |
| | STYYADN | |
| | VLGRFTISRDNSKNTLYLQMNSLRAEDTAVYYCARTSSGYFDYWGQGTLVTVSS | |
| VL | SSVLTQPPSVSGAPGQRVTISCSGSSSNIGSNTVNWYQQLPGTAPKLLIYGNNNRPSG | |
| | VPDRFS | |
| | GSKSGTSASLAITGLQSEDEADYYCAAWDDSLNGVVFGGGTKLTVL | |
| | VL V96 | 87.6 |
| | VL W96 | 88.6 |
| | VL Y96 | 88.9 |
| | VL F96 | 88.9 |
| | VL H96 | 88.3 |
| | VL M96 | 87.2 |
| | VL L96 | 85.9 |
| | VL I96 | 87.8 |
| | VL P96 | 86.2 |
| Example 25 | | |
| VH | EVQLVESGGGLVQPGGSLRLSCAASGFTFSSNYMSWVRQAPGKGLEWVGTISGSGG | |
| | STYYADN | |
| | VLGRFTISRDNSKNTLYLQMNSLRAEDTAVYYCARTSSGYFDYWGQGTLVTVSS | |
| VL | SSVLTQPPSVSGAPGQRVTISCSGSSSNIGSNTVNWYQQLPGTAPKLLIYGNNNRPSG | |
| | VPDRFS | |
| | GSKSGTSASLAITGLQSEDEADYYCAAWDDSLNGYVFGGGTKLTVL | |
| | VL N34 | 88.9 |
| | VL S34 | 91.8 |
| Example 26 | | |
| VH | EVQLVESGGGLVQPGGSLRLSCAASGFTFSSNYMSWVRQAPGKGLEWVGTISGSGG | |
| | STYYADNV | |
| | LGRFTISRDNSKNTLYLQMNSLRAEDTAVYYCARTSSGYFDYWGQGTLVTVSS | |
| VL | SSVLTQPPSVSGAPGQRVTISCSGSSSNIGSNTVNWYQQLPGTAPKLLIYGNNNRPSG | |
| | VPDRFSGS | |
| | KSGTSASLAITGLQSEDEADYYCAAWDDSLNGYVFGTGTKLTVL | |
| | VL N34 | 89.1 |
| | VL S34 | 92 |
| Example 27 | | |
| VH | EVQLVESGGGLVQPGGSLRLSCAASGFTFSSNYMSWVRQAPGKGLEWVGTISGSGG | |
| | STYYADNV | |
| | LGRFTISRDNSKNTLYLQMNSLRAEDTAVYYCARTSSGYFDYWGQGTLVTVSS | |
| VL | SSVLTQPPSVSGAPGQRVTISCSGSSSNIGSNTVNWYQQLPGTAPKLLIYGNNNRPSG | |
| | VPDRFSGS | |
| | KSGTSASLAITGLQSEDEADYYCAAWDDSLNGYVFGGGTKLTVL | |
| | VL G100 | 88.9 |
| | VL T100 | 89.1 |
| Example 28 | | |
| VH | EVQLVESGGGLVQPGGSLRLSCAASGFTFSSNYMSWVRQAPGKGLEVVVGTISGSGG | |
| | STYYADN | |
| | VLGRFTISRDNSKNTLYLQMNSLRAEDTAVYYCARTSSGYFDYWGQGTLVTVSS | |
| VL | SSVLTQPPSVSGAPGQRVTISCSGSSSNIGSNTVSWYQQLPGTAPKLLIYGNNNRPSG | |
| | VPDRFSG | |
| | SKSGTSASLAITGLQSEDEADYYCAAWDDSLNGYVFGGGTKLTVL | |
| | VL G100 | 91.8 |
| | VL T100 | 92 |

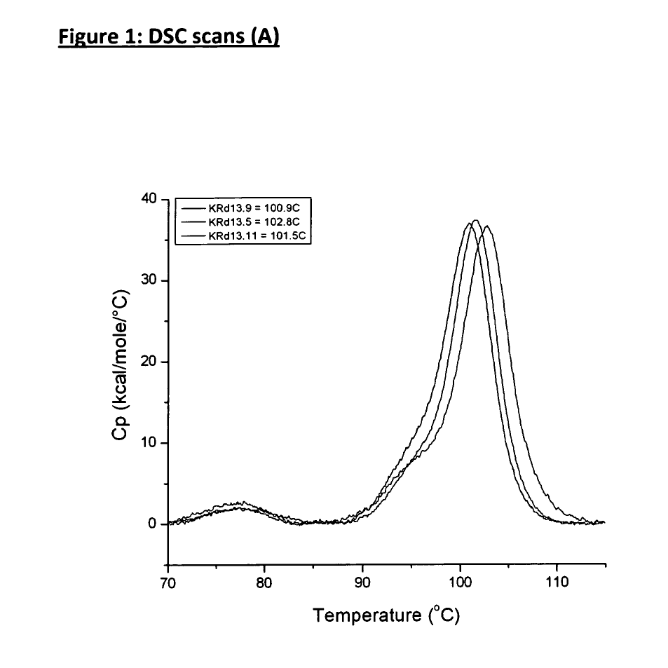

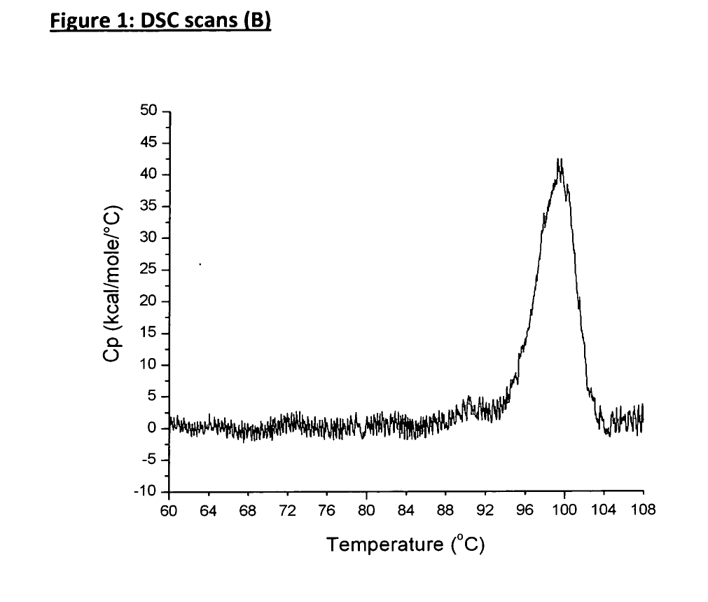

FIG. 1 shows an exemplary graph of a typical DSC scan.

### Example 4

### Human Fab Fragment with a Melting Temperature Above 100° C.

Combining several of the improved sequence features identified in this invention allows generation of exceptionally thermostable Fab fragments. In some instances, melting temperatures of such combination mutants can exceed 100° C., even at a slow scan rate of 1° C. per min. For example, antibody clone KRd15.6 with the VH domain amino acid sequence EAQLVESGGGLVQPGGSLRLSCAASGFTFSSYAMSWIRQAPGKGLEWIGQIS GSGGSTYYNDSVKGRFTISRDNSKNTLYLQMNSLRPEDTAVYYCARDSGYFD IWGQGTLVTVSS and the VK domain amino acid sequence AIQMTQSPSSLSASVGDRVTITCRASQSISSYLAWYQQKPGKAPKLLIYAASSL YSGVPSRFSGSGSGTDFTLTISSLQPEDFATYYCQQYSSLPYTFGQGTKVEIK R was analysed as follows.

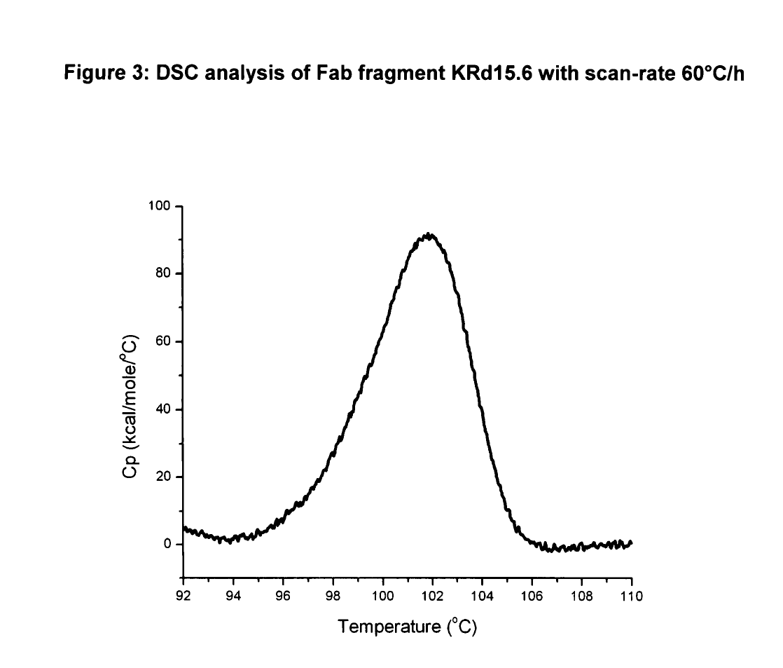

The Fab fragment was expressed in E. coli, affinity-purified on protein A resin and buffer-exchanged into 1×PBS pH 7.4 (Invitrogen catalogue No. 10010056, containing 1.06 mM KH₂PO₄, 2.97 mM Na₂HPO₄×7H₂O, 155.17 mM NaCl and no other supplements). For DSC measurements, a capillary cell microcalorimeter equipped with autosampler and controlled by VPViewer2000 CapDSC software from MicroCal was used. The Fab fragment was scanned against pure buffer containing no antibody (1×PBS pH 7.4; Invitrogen catalogue No. 10010056). The scan parameters were set to analyze a temperature window from 32° C. to 115° C., with a pre-scan thermostat of 2 min, a post-scan thermostat of 0 min and no gain. The scan rate was set to 60° C. per h. FIG. 3 shows the graph of the DSC scan. The data analysis was performed using Origin 7.0 software from MicroCal and was automated to avoid any subjective user input. First, pure PBS scanned against pure PBS was used for reference subtraction. Second, the scan was normalized for protein concentration using the absorbance determined at 280 nm and the calculated extinction coefficient of the Fab fragment. Third, the displayed data range was set to be 55° C. to 115° C. Fourth, the baseline was subtracted using the “cubic connect” function. Finally, data were fitted to the Non-2-State model, using 200 Levenberg-Marquardt iterations. The melting temperature of the full intact Fab fragment in the given example is 101.5° C.±0.0066° C., clearly above boiling point and far above the previously highest melting temperature previously published for a Fab fragment of 92° C. (Demarest et al., 2006).

TABLE 2
Accession Numbers

| Accession | |
|---|---|
| No. | Sequence |
| AJ704539 | QVQLQQSGADLKVPGASVKVSCKSSGYWFHDYAALALGRAPGKGLEWTGWIN |
| | TNYGETNYAQKFLGGVTMTRDKSTSTGTELIRLGSDDTAVYYCARLIVSDRYGQ |
| | GTMVTASSGGGGSSGGGGSGGSALAIQMTQSPSSLSASVGDRVTITCRASQGI |
| | RNDLGVVYQQKPGKAPKLLIYAASSLQSGVPSRFSGSGSGTDFILTISSLQPED |
| | FATYYCQQYNSYHTFGQGTKVEIKRAAAHHHHHH |
| U43767 | MDMGAHVHLLGLLLLWLPGARCAIQMTQSPSSLSASVGDRVTITCRASQGIRND |
| | LGWYQQKPGKAPKLLIYAASSLQSGVPSRFSGSRSGTDFTLTISSLQPEDFATYY |
| | CLRDYNYSVVTFGQGTKVEIKRTVAAPSVFIFPPSDEAW |
| 4762 | AIQMTQSPSSLSASVGDRVTITCRASQGIRNDLGWYQQKPGKAPKLLIYAASSL |
| | QSGVPSRFSGSGSGTDFTLTISSLQPEDFATYYCLQDYNYP |
| 40096 | AIQMTQSPSSLSASVGDRVTITCRASQGIRNDLGWYQQKPGKAPKLLIYAASS |
| | LQSGVPSRFSGSRSGTDFTLTISSLQPEDFATYYCLRDYNYSWTFGQGTKVEI |
| | KRT |
| 21224 | AIQMTQSPSSLSASVGDRVTITCRASQGIRNDLGWYQQKPGKAPKRLIYAAFIW |
| | QSGVPSRFSGSGSGTEFTLTISSLQPEDFATYYCLQHNSFPYTFGQGTKLEVKR |
| CS483741 | AIQLTQSPSSLSASVGDRVTITCRASQGISSALAWYQQKPGKAPKLLIYDASSL |
| | ESGVPSRFSGSGSGTDFTLTISSLQPEDFATYYCQQFNSYPYTFGQGTKLEIK |
| CS483744 | AIQLTQSPSSLSASVGDRVTITCRASQGISSALAWYQQKPGKAPKLLIYDASSL |
| | ESGVPSRFSGSGSGTDFTLTISSLQPEDFATYYCQQFNSYPYTFGQGTKLEIK |
| U86790 | MDMRVPAQLLGLLLLWLPGARCAIQLTQSPSSLSASVGDRVTITCRASQGISS |
| | ALAWYQQKPGKAPKLLIYDASSLESGVPSRFSGSGSGTDFTLTISSLQPEDFA |
| | TYYCQQFNIFGGGTKVEIKRIRAR |
| X72459 | PAQLLGLLLLWLPGARCAIQLTQSPSSLSASVGDRVTITCRASQGISSALAWY |
| | QQKPGKAPKWYDASSLESGVPSRFSGSGSGTDFTLTISSLQPEDFATYYCQQ |
| | FNTYPLTFGGGTKVEIKR |
| 4753 | AIQLTQSPSSLSASVGDRVTITCRASQGISSALAWYQQKPGKAPKLLIYDASSL |
| | ESGVPSRFSGSGSGTDFTLTISSLQPEDFATYYCQQFNSYP |
| 19244 | AIQLTQSPSSLSASVGDRVTITCRASQGISSALAWYQQKPGKAPKLLIYDASSL |
| | ESGVPSRFSGSGSGTDFTLTISSLQPEDFATYYCQQFNTYPLTFGGGTKVEIKR |
| AY043163 | AIQLTQSPSSLSASVGDRVTITCRASQGITSRSAWYQQKPGKAPRLLIYGVSNL |
| | ESGVPSRFSGSASGTDFTLTISSLQPEDFATYYCQQINNSPAFGQGTRLEIK |
| L26891 | LGLLLLWLPGARCAIQMTQSPSSLSASVGDRVTITCRASQDIRNDLGWYQQQP |
| | GKAPKLLIYAASTLHTGVPSRFSGSRSGTTFTLTISGLQPEDFATYYCLQDYNY |
| | VVTFGQGTRVEIKRTVAAPSVF |
| DQ184511 | DTQMTQSPSSLSASVGDRVTITCRASQSISSYLNWYQQKPGKAPKLLIYYASY |
| | LQSGVPSRFSGSGSGTDFTLTISSLQPEDFATYYCQQTYTAPDTFGQGTKVEIKR |
| AY686924 | MAETTLTQSPSSLSASVGDRVTITCRASQSISSYLNWYQQKPGKAPKLLIYAA |
| | SSLQSGVPSRFSGSGSGTDFTLTISSLQPEDFATYYCQQSYSTPHTFGQGTKVEI |
| | K |
| 4806 | DTQLTQSPSFLSASVGDRVTITCRASQGISSYLAWYQQKPGKAPRLLIYATST |
| | LQSGVPSRFSGSGSGTEFTLTISSLQPEDFATYYCQQLNSYPPYTFGQGTKLEIN |
| DQ535161 | PLTSMTQSPSSLSASIGDRVTITCRASQSISIFLNWFQQRPGKAPKLLIYAASSL |
| | QGGVPSRFSGSGSGTDFTLTITSLQPEDFATYYCQQSFSIPVVTFGQGTNVDIK |
| 1s78_C | diqmtqspsslsasvgdrvtitckasqdvsigvawyqqkpgkapklliysasyrytgvpsrfsgsg |
| | sgtdftltisslqpedfatyycqqyyiypytfgqgtkveikrtvaapsvfifppsdeqlksgtasvvclinn |
| | fypreakvqwkvdnalqsgnsqesvteqdskdstyslsstltlskadyekhkvyacevthqgls |
| | spvtksfnrgec |
| 1s78_E | diqmtqspsslsasvgdrvtitckasqdvsigvawyqqkpgkapklliysasyrytgvpsrfsgs |
| | gsgtdftltisslqpedfatyycqqyyiypytfgqgtkveikrtvaapsvfifppsdeqlksgtasvvc |
| | llnnfypreakvqwkvdnalqsgnsqesvteqdskdstyslsstitlskadyekhkvyacevthqglsspvtksfnr |
| | gec |
| 1l7i_L | diqmtqspsslsasvgdrvtitckasqdvsigvawyqqkpgkapklliysasyrytgvpsrfsgs |
| | gsgtdffitisslqpedfatyycqqyyiypytfgqgtkveikrtvaapsvfifppsdeqlksgtasvvc |
| | llnnfypreakvqwkvdnalqsgnsqesvteqdskdstysisstltlskadyekhkvyacevth |
| | qglsspvtksfnrgec |
| AM082547 | VQCEAQLLESGGGLVQRGGSLRLSCAASGFTFSNYAMSWVRQAPGKGLE |
| | WVSTTSGSGASTYHADSVKGRFTISRDNSKNTLYLQMNSLRAEDTAVYYC |
| | AK |
| AM082383 | VQCEAQLLESGGGLVQPGGSLRLSCAASGFTFTTYAMSWVRQAPGKGLE |
| | WVSTITGGGGGTDYADSVKGRFTISRDNSKNTLYLQMNSLRAEDTAVYYC |
| | AK |
| AM080583 | VQCEVQLLESGGGLVQPGGSLRLSCAASGFTFSNFAMSWIRQAPGKGLEW |
| | VSTLSGGGGSTYYADSVKGRFTISRDNSKNTLYLQMNSLRAEDTAVYHCG |
| | K |
| AF471288 | EVQLVESGGGLVQPGGSLRLSCVASGFTFTSYAMIWVRQAPGKGLEWISTI |
| | NDSGGRTYYADSVKGRFTVSRDNSKNTLYLQMNSLRAEDSAVYYCVNDK |
| | ERDDGGWRDPWGQGTLVTVSS |
| AM082399 | VQCEVQLLESGGGLVQPGGSLRLSCAASGFTFSNYPMSWIRQAPGKGLEW |
| | VSTLSGSGVTTFYADSGKGRFTISRDNSKNTLYLQMSSLRADDTAVYYCA |
| | K |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

To the extent possible under the respective patent law, all patents, applications, publications, test methods, literature, and other materials cited herein are hereby incorporated by reference.

### REFERENCES

Aburatani T, Ueda H, Nagamune T.
Importance of a CDR H3 basal residue in V(H)/V(L) interaction of human antibodies.
J Biochem. 2002 November; 132(5):775-82.
Al-Lazikani B, Lesk A M, Chothia C.
J Mol Biol. 1997 Nov. 7; 273(4):927-48.
Standard conformations for the canonical structures of immunoglobulins.
Arndt K M, Müller KM, PlÜckthun A.
Helix-stabilized Fv (hsFv) antibody fragments: substituting the constant domains of a Fab fragment for a heterodimeric coiled-coil domain.
J Mol Biol. 2001 Sep. 7; 312(1):221-8.
Barthelemy P A, Raab H, Appleton B A, Bond C J, Wu P, Wiesmann C, Sidhu S S.
Comprehensive analysis of the factors contributing to the stability and solubility of autonomous human VH domains.
J Biol Chem. 2008 Feb. 8; 283(6):3639-54. Epub 2007 Nov. 28.
Carter P, Presta L, Gorman C M, Ridgway J B, Henner D, Wong W L, Rowland A M, Kotts C, Carver M E, Shepard H M.
Humanization of an anti-p185HER2 antibody for human cancer therapy.
Proc Natl Acad Sci USA. 1992 May 15; 89(10):4285-9.
Chothia C, Lesk A M, Gherardi E, Tomlinson I M, Walter G, Marks J D, Llewelyn M B, Winter G.
Structural repertoire of the human VH segments.
J Mol Biol. 1992 Oct. 5; 227(3):799-817.
Davies J, Riechmann L.
‘Camelising’ human antibody fragments: NMR studies on VH domains.
FEBS Lett. 1994 Feb. 21; 339(3):285-90.
Davies J, Riechmann L.
Single antibody domains as small recognition units: design and in vitro antigen selection of camelized, human VH domains with improved protein stability. Protein Eng. 1996 June; 9(6):531-7.
de Wildt R M, Hoet R M, van Venrooij W J, Tomlinson I M, Winter G.
Analysis of heavy and light chain pairings indicates that receptor editing shapes the human antibody repertoire.
J Mol Biol. 1999 Jan. 22; 285(3):895-901.
Demarest S J, Chen G, Kimmel B E, Gustafson D, Wu J, Salbato J, Poland J, Elia M, Tan X, Wong K, Short J, Hansen G.
Engineering stability into Escherichia coli secreted Fabs leads to increased functional expression.
Protein Eng Des Sel. 2006 July; 19(7):325-36. Epub 2006 May 3.
Dottorini T, Vaughan C K, Walsh M A, LoSurdo P, Sollazzo M.
Crystal structure of a human VH: requirements for maintaining a monomeric fragment.
Biochemistry. 2004 Jan. 27; 43(3):622-8.
Dudgeon K, Famm K, Christ D.
Sequence determinants of protein aggregation in human VH domains.
Protein Eng Des Sel. 2009 March; 22(3):217-20. Epub 2008 Oct. 28.
(A) Ewert S, Huber T, Honegger A, Plückthun A.
Biophysical properties of human antibody variable domains.
J Mol Biol. 2003 Jan. 17; 325(3):531-53.
(B) Ewert S, Honegger A, Plückthun A.
Structure-based improvement of the biophysical properties of immunoglobulin VH domains with a generalizable approach.
Biochemistry. 2003 Feb. 18; 42(6):1517-28.
Fellouse F A, Li B, Compaan D M, Peden A A, Hymowitz S G, Sidhu S S.
Molecular recognition by a binary code.
J Mol Biol. 2005 May 20; 348(5):1153-62. Epub 2005 Apr. 1.
Fellouse F A, Barthelemy P A, Kelley R F, Sidhu S S.
Tyrosine plays a dominant functional role in the paratope of a synthetic antibody derived from a four amino acid code.
J Mol Biol. 2006 Mar. 17; 357(1):100-14. Epub 2005 Dec. 19.
Fellouse F A, Esaki K, Birtalan S, Raptis D, Cancasci V J, Koide A, Jhurani P, Vasser M, Wiesmann C, Kossiakoff A A, Koide S, Sidhu S S.
High-throughput generation of synthetic antibodies from highly functional minimalist phage-displayed libraries.
J Mol Biol. 2007 Nov. 2; 373(4):924-40. Epub 2007 Aug. 19.
Garber E, Demarest S J.
A broad range of Fab stabilities within a host of therapeutic IgGs.
Biochem Biophys Res Commun. 2007 Apr. 13; 355(3):751-7. Epub 2007 Feb. 16.
Hoet R M, Cohen E H, Kent R B, Rookey K, Schoonbroodt S, Hogan S, Rem L, Frans N, Daukandt M, Pieters H, van Hegelsom R, Neer N C, Nastri H G, Rondon I J, Leeds J A, Hufton S E, Huang L, Kashin I, Devlin M, Kuang G, Steukers M, Viswanathan M, Nixon A E, Sexton D J, Hoogenboom H R, Ladner R C.
Generation of high-affinity human antibodies by combining donor-derived and synthetic complementarity-determining-region diversity.
Nat Biotechnol. 2005 March; 23(3):344-8. Epub 2005 Feb. 20.
Honegger A.
Engineering antibodies for stability and efficient folding.
Handb Exp Pharmacol. 2008; (181):47-68. Review.
(A) Jespers L, Schon O, Famm K, Winter G.
Aggregation-resistant domain antibodies selected on phage by heat denaturation.
Nat Biotechnol. 2004 September; 22(9):1161-5. Epub 2004 Aug. 8.
(B) Jespers L, Schon O, James L C, Veprintsev D, Winter G.
Crystal structure of HEL4, a soluble, refoldable human V(H) single domain with a germ-line scaffold.
J Mol Biol. 2004 Apr. 2; 337(4):893-903.
Jung S, Plückthun A.
Improving in vivo folding and stability of a single-chain Fv antibody fragment by loop grafting.
Protein Eng. 1997 August; 10(8):959-66.
Jung S, Honegger A, Plückthun A.
Selection for improved protein stability by phage display.
J Mol Biol. 1999 Nov. 19; 294(1):163-80.
Kabat E A, Wu T T.
Identical V region amino acid sequences and segments of sequences in antibodies of different specificities. Relative contributions of VH and VL genes, minigenes, and complementarity-determining regions to binding of antibody-combining sites.
J Immunol. 1991 Sep. 1; 147(5):1709-19.
Knappik A, Ge L, Honegger A, Pack P, Fischer M, Wellnhofer G, Hoess A, Wölle J, Plückthun A, Virnekäs B.
Fully synthetic human combinatorial antibody libraries (HuCAL) based on modular consensus frameworks and CDRs randomized with trinucleotides.
J Mol Biol. 2000 Feb. 11; 296(1):57-86.
Lee C V, Liang W C, Dennis M S, Eigenbrot C, Sidhu S S, Fuh G.
High-affinity human antibodies from phage-displayed synthetic Fab libraries with a single framework scaffold.
J Mol Biol. 2004 Jul. 23; 340(5):1073-93.
Lefranc M P, Giudicelli V, Ginestoux C, Bodmer J, Müller W, Bontrop R, Lemaitre M, Malik A, Barbié V, Chaume D.
IMGT, the international ImMunoGeneTics database.
Nucleic Acids Res. 1999 Jan. 1; 27(1):209-12.
Martineau P, Betton J M.
In vitro folding and thermodynamic stability of an antibody fragment selected in vivo for high expression levels in Escherichia coli cytoplasm.
J Mol Biol. 1999 Oct. 1; 292(4):921-9.
Muller Y A, Heiring C, Misselwitz R, Welfle K, Welfle H.
The cystine knot promotes folding and not thermodynamic stability in vascular endothelial growth factor.
J Biol Chem. 2002 Nov. 8; 277(45):43410-6.
Nieba L, Honegger A, Krebber C, Plückthun A.
Disrupting the hydrophobic patches at the antibody variable/constant domain interface: improved in vivo folding and physical characterization of an engineered scFv fragment.
Protein Eng. 1997 April; 10(4):435-44.
Ohage E C, Graml W, Walter M M, Steinbacher S, Steipe B.
Beta-turn propensities as paradigms for the analysis of structural motifs to engineer protein stability.
Protein Sci. 1997 January; 6(1):233-41.
Ohage E, Steipe B.
Intrabody construction and expression. I. The critical role of VL domain stability.
J Mol Biol. 1999 Sep. 3; 291(5):1119-28.
Persson M A.
Twenty years of combinatorial antibody libraries, but how well do they mimic the immunoglobulin repertoire?
Proc Natl Acad Sci USA. 2009 Dec. 1; 106(48):20137-8. Epub 2009 Nov. 23. No abstract available.
Proba K, Wörn A, Honegger A, Plückthun A.
Antibody scFv fragments without disulfide bonds made by molecular evolution.
J Mol Biol. 1998 Jan. 16; 275(2):245-53.
Retter I, Althaus H H, Münch R, Müller W.
VBASE2, an integrative V gene database.
Nucleic Acids Res. 2005 Jan. 1; 33(Database issue):D671-4.
Ribnicky B, Van Blarcom T, Georgiou G.
A scFv antibody mutant isolated in a genetic screen for improved export via the twin arginine transporter pathway exhibits faster folding.
J Mol Biol. 2007 Jun. 8; 369(3):631-9. Epub 2007 Mar. 31.
Riechmann L.
Rearrangement of the former VL interface in the solution structure of a camelised, single antibody VH domain.
J Mol Biol. 1996 Jun. 28; 259(5):957-69.
Rothe C, Urlinger S, Löhning C, Prassler J, Stark Y, Jäger U, Hubner B, Bardroff M, Pradel I, Boss M, Bittlingmaier R, Bataa T, Frisch C, Brocks B, Honegger A, Urban M.
The human combinatorial antibody library HuCAL GOLD combines diversification of all six CDRs according to the natural immune system with a novel display method for efficient selection of high-affinity antibodies.
J Mol Biol. 2008 Feb. 29; 376(4):1182-200. Epub 2007 Dec. 15.
Silacci M, Brack S, Schirru G, M\dot{a}rlind J, Ettorre A, Merlo A, Viti F, Neri D.
Design, construction, and characterization of a large synthetic human antibody phage display library.
Proteomics. 2005 June; 5(9):2340-50.
Steipe B, Schiller B, Plückthun A, Steinbacher S.
Sequence statistics reliably predict stabilizing mutations in a protein domain.
J Mol Biol. 1994 Jul. 15; 240(3):188-92.
Sterner R, Liebl W.
Thermophilic adaptation of proteins.
Crit Rev Biochem Mol Biol. 2001; 36(1):39-106.
Tan P H, Sandmaier B M, Stayton P S.
Contributions of a highly conserved VH/VL hydrogen bonding interaction to scFv folding stability and refolding efficiency.
Biophys J. 1998 September; 75(3):1473-82.
Tanha J, Xu P, Chen Z, Ni F, Kaplan H, Narang S A, MacKenzie C R.
Optimal design features of camelized human single-domain antibody libraries.
J Biol Chem. 2001 Jul. 6; 276(27):24774-80. Epub 2001 May 2.
Tomlinson I M, Cox J P, Gherardi E, Lesk A M, Chothia C.
The structural repertoire of the human V kappa domain.
EMBO J. 1995 Sep. 15; 14(18):4628-38.
Wetzel S K, Settanni G, Kenig M, Binz H K, Plückthun A.
Folding and unfolding mechanism of highly stable full-consensus ankyrin repeat proteins.
J Mol Biol. 2008 Feb. 8; 376(1):241-57.
Wörn A, Plückthun A.
Stability engineering of antibody single-chain Fv fragments.
J Mol Biol. 2001 Feb. 2; 305(5):989-1010. Review.

1. A method for modifying a parental antibody variable domain comprising a variable heavy (VH) chain domain and a variable light (VL) chain domain, comprising the steps of
(a) establishing a structural model of said parental antibody variable domain based on its amino acid sequence;
(b) identifying in the six CDR regions of the VH and VL chain domains one or more CDR amino acid residues, which are buried in the interface between the VH domain and the VL domain, and which are not determinants of a specific canonical structure;
(c) replacing at least one of the amino acid residues identified in step (b) by a different amino acid residue to generate one or more antibody variable domain variants;
(d) optionally replacing in step (c) one or more additional amino acid residues in the CDR regions and/or in the framework regions of said parental antibody variable domain.

2. The method of claim 1, wherein steps (c) and optionally (d) are performed by modifying one or more nucleic acid sequences encoding the parental antibody variable domain.

3. The method of claim 2, comprising the additional step of:
(e) expressing the one or more nucleic acid sequences encoding each of said one or more antibody variable domain variants.

4. The method of claim 3, comprising the additional steps of:
(f) comparing the stability of said one or more antibody variable domain variants with the stability of the parental antibody; and
(g) selecting an antibody variable domain variant with improved stability.

5. The method of claim 4, comprising the additional step of:
(h) repeating steps (c) to (g) one or more times by using the antibody variable domain variant selected in the previous step (g) as new parental antibody variable domain in step (c).

6. The method of any of claim 1, wherein in step (c) or (d) at least one amino acid residue is changed from an amino acid being the consensus amino acid for that position in the family of antibody sequences the parental antibody variable domain belongs to a non-consensus amino acid.

7. A method for modifying a parental antibody variable domain, comprising the step of:
(i) making or causing in a parental Vkappa1 antibody variable domain one or more of the following changes:
(a) in the CDR regions:
(aa) at position L55 a change to an amino acid selected from Y, H, and W, particularly to Y;
(ab) at position L94 a change to an amino acid selected from F, H, I, K, L, M, R, T, V, and Y, particularly L;
(ac) at position L96 a change to an amino acid selected from F and Y, particularly Y;
(ad) at position L32 a change to an amino acid selected from D, F, K, N, Q, S, and Y;
(ae) at position L34 a change to an amino acid selected from A,S, and T, particularly A and S, particularly A;
(af) at position L91 a change to an amino acid selected from A, G, S, and Y, particularly Y; and/or
(b) in the framework regions:
(ba) at position L1 a change to amino acid A;
(bb) at position L2 a change to amino acid T; and/or
(bc) at position L70 a change to amino acid E; or
(ii) making or causing in a parental Vlambda1 antibody variable domain one or more of the following changes:
(a) in the CDR regions:
(aa) at position L34 a change to an amino acid selected from G and S, particularly S;
(ab) at position L96 a change to an amino acid selected from F and Y, particularly to Y; and/or
(ac) at position L100 a change to amino acid T; and/or
(iii) making or causing in a parental VH3 antibody variable domain one or more of the following changes:
(a) in the CDR regions:
(aa) at position H50 a change to an amino acid selected from Q, S and T, particularly S and T, particularly S, when said VH3 antibody variable domain is combined with a Vkappa antibody variable domain;
(ab) at position H60 a change to amino acid N;
(ac) at position H63 a change to an amino acid selected from V, I, and F;
(ad) at position H64 a change to amino acid L;
(ae) at position H95 a change to amino acid selected from D, N and T, particularly to D;
(af) at position H102 a change to an amino acid selected from I and V;
(ag) at position H28 a change to amino acid P;
(ah) at position H33 a change to amino acid A;
(ai) at position H52 a change to an amino acid selected from D and S, particularly to D;
(aj) at position H(103 minus 5) a change to amino acid G;
(aj) one or two changes at positions H50 and H95 in order to create a salt bridge, particularly the following salt bridges: H50:R/H95:E; and H50:H/H95:E;
(ak) one or two changes at positions H33 and H95 in order to create a salt bridge, particularly the following salt bridges: H33:R/H95:E; H33:R/H95:D; H33:H/H95:D; and H33:D/H95:H; and/or
(b) in the framework regions:
(ba) at position H2 a change to an amino acid selected from A and G;
(bb) at position H37 a change to amino acid I;
(bc) at position H48 a change to amino acid I; and/or
(bd) at position H49 a change to amino acid G.

8. The method of claim 7, wherein said parental antibody variable domain is modified by making or causing at least one of the changes listed in (i)(a), (ii)(a) and (iii)(a).

9. The method of claim 7 or 8, wherein at least two of said changes are made or caused, particularly wherein at least three of said changes are made or caused.

10. An antibody variable domain comprising at least one VL or VH domain selected from the group of:
(i) a Vkappa1 antibody variable domain based on the antibody variable domain of SEQ ID No. 1, comprising one or more of the following changes:
(A) a single amino acid exchange L2:I to L2:T; or
(B) at least two amino acid changes independently selected from the following group:
(a) in the CDR regions:
(aa) L55:Q to L55:Y;
(ab) L94:T to L941; and/or
(ac) L96:L to L96:Y; and/or
(b) in the framework regions:
(ba) L1:D to L1:A;
(bb) L2:I to L2:T; and/or
(bc) L70:D to L70:E;
and optionally comprising up to 3 additional changes in the framework regions FR1 to FR3 different from those of (i)(A) and/or (B);
provided that the antibody variable domains having the following accession numbers are excluded: AJ704539, U43767, 4762, 40096, 21224, CS483741, CS483744, U86790, X72459, 4753, 19244, AY043163, L26891, DQ184511, AY686924, 4806, DQ535161, 1S78_C, 1S78_E, and 1L7I_L;
(ii) a Vlambda1 antibody variable domain based on the antibody variable domain of SEQ ID No. 2, comprising the following combination of changes:
(a) in the CDR regions:
(aa) L34:N to L34:S; and
(ab) L96:V to L96:Y or L96:V to L96:F;
and optionally further comprising up to 3 additional changes in the framework regions FR1 to FR3 different from those of (ii)(a);
(iii) a VH3 antibody variable domain based on the antibody variable domain of SEQ ID No. 3, comprising one or more of the following changes:
(A) a single amino acid exchange selected from the following group:
(a) in the CDR regions:
(aa) H50V: to H50:T;
(ab) H60A: to H60:N;
(ac) H63V: to H63:I
(ad) H63V: to H63:F; and
(ae) H64:K to H64:L, provided that H:63 is not D;
(B) at least two amino acid changes independently selected from the following group:
(a) in the CDR regions:
(aa) H50V: to H50:Q;
(ab) H50V: to H50:T;
(ac) H60A: to H60:N;
(ad) H63V: to H63:I
(ae) H63V: to H63:F;
(af) H64:K to H64:L, provided that H:63 is not D; and
(ag) H95:D to H95: N; and/or
(b) in the framework regions:
(ba) H2:V to H2:A;
(bb) H37:V to H37:I;
(bc) H48:V to H48:I; and/or
(bd) H49:S to H49:G;
in both (A) and (B) provided that the antibody variable domains having the following accession numbers are excluded: AM082547, AM082383, AM080583, AF471288, and AM082399.

11. A method for modifying an antibody variable domain, comprising the step of:
(i) making or causing in a Vkappa1 antibody variable domain having a sequence identity in the framework regions FR1 to FR3 of at least 90% to the antibody variable domain of SEQ ID No. 1 one or more of the following changes:
(a) in the CDR regions:
(aa) L55:Q to L55:(Y,H,W), particularly L55:Y;
(ab) L94:T to L94:(F, H, I, K, L, M, R, T, V, Y), particularly L941; and/or
(ac) L96:L to L96:(F,Y);
(ad) L32:Y to L32(D, F, K, N, Q, S); and/or
(b) in the framework regions:
(ba) L1:D to L1:(A,D), particularly L1:A;
(bb) L2:I to L2:T; and/or
(bc) L70:D to L70:E;
(ii) making or causing in a Vlambda1 antibody variable domain having a sequence identity in the framework regions FR1 to FR3 of at least 90% to the antibody variable domain of SEQ ID No. 2 one or more of the following changes:
(a) in the CDR regions:
(aa) L34:N to L34:S;
(ab) L96:V to L96:Y; and/or
(ac) L96:V to L96:F; and/or
(iii) making or causing in a VH3 antibody variable domain having a sequence identity in the framework regions FR1 to FR3 of at least 90% to the antibody variable domain of SEQ ID No. 3 one or more of the following changes:
(a) in the CDR regions:
(aa) H50:V to H50:Q;
(ab) H50:V to H50:T;
(ac) H60:A to H60:V;
(ad) H63:V to H63:I
(ae) H63:V to H63:F
(af) H63:V to H63:Q and/or
(ag) H64:K to H64:L, provided that H:63 is not D; and
(ah) H95:D to H95: N;
(ai) H50:V to H50:S, particularly when said VH3 antibody variable domain is combined with a Vkappa antibody variable domain;
(aj) H28:T to H28:P;
(ak) H52:S to H52:D;
(al) H(103-5):X to H(103-5):G;
(am) H50/H95 to a salt bridge, particularly a salt bridge selected from: H50:R/H95:E; and H50:H/H95:E;
(an) H33/H95 to a salt bridge, particularly a salt bridge selected from: H33:R/H95:E; H33:R/H95:D; H33:H/H95:D; and H33:D/H95:H; and/or
(b) in the framework regions:
(ba) H2:V to H2:A;
(bb) H37:V to H37:I;
(bc) H48:V to H48:I; and/or
(bd) H49:S to H49:G.

12. A method of using of an antibody variable domain according to the method of claim 1 or 2, in the construction of a diverse collection of antibody variable domains, comprising the step of:
(a) diversifying one or more amino acid positions in one or more CDR regions of said antibody variable domain, provided that
(aa) none of the following CDR positions is diversified: Vkappa1: L96; Vlambda1: L96; VH3: H50 and H95; and the following CDR positions are each independently optionally diversified: Vkappa1: L55 and L94; VH3: H60, H63, and H64; or
(ab) any of the following CDR positions is not diversified, if it carries one of the following amino acid residues: Vkappa1: L55:Y, L96:Y; Vlambda1: L96:Y; VH3: H50:T, H60:N, H63:I, H64:L, and H95:D; or
(ac) any of the following CDR positions is either not diversified, or it is diversified with a bias towards the following amino acid residues: Vkappa1: L55:Y, L96:Y; Vlambda1: L96:Y; VH3: H50:T, H60:N, H63:I, H64:L, and H95:D; particularly wherein the listed amino acid residues is present to at least 30%, and more particularly to at least 50% in the diversification mixture; or
(ad) any of the following CDR positions is either not diversified or diversified with the indicated limited diversity only: Vkappa1: L55:YHW, L94:FHIKLRY, L96:FY; Vlambda1: L96:FY; VH3: H50:QT, H60:HNRS, H63:VIF, H64:KL, and H95:DNT.

13. A method for construction of a diverse collection of antibody variable domains, comprising the step of (a) diversifying one or more amino acid positions in one or more CDR regions of an antibody variable domain according to the method of claim 1 or 2, provided that
(aa) none of the following CDR positions is diversified: Vkappa1: L96; Vlambda1: L96; VH3: H50 and H95; and the following CDR positions are each independently optionally diversified: Vkappa1: L55 and L94; VH3: H60, H63, and H64; or
(ab) any of the following CDR positions is not diversified, if it carries one of the following amino acid residues: Vkappa1: L55:Y, L96:Y; Vlambda1: L96:Y; VH3: H50:T, H60:N, H63:I, H64:L, and H95:D; or
(ac) any of the following CDR positions is either not diversified, or it is diversified with a bias towards the following amino acid residues: Vkappa1: L55:Y, L96:Y; Vlambda1: L96:Y; VH3: H50:T, H60:N, H63:I, H64:L, and H95:D; particularly wherein the listed amino acid residues is present to at least 30%, and more particularly to at least 50% in the diversification mixture; or
(ad) any of the following CDR positions is either not diversified or diversified with the indicated limited diversity only: Vkappa1: L55:YHW, L94:FHIKLRY, L96:FY; Vlambda1: L96:FY; VH3: H50:QT, H60:HNRS, H63:VIF, H64:KL, and H95:DNT.

14. A diverse collection of antibody variable domains, wherein said collection comprises one or more diverse collections of amino acid residues at one or more positions in one or more CDR regions, provided that
(aa) none of the following CDR positions is diversified: Vkappa1: L96; Vlambda1: L96; VH3: H50 and H95; and the following CDR positions are each independently optionally diversified: Vkappa1: L55 and L94; VH3: H60, H63, and H64; or
(ab) any of the following CDR positions is not diversified, if it carries one of the following amino acid residues: Vkappa1: L55:Y, L94:L, L96:Y; Vlambda1: L96:Y; VH3: H50:T, H60:N, H63:I, H64:L, and H95:D; or
(ac) any of the following CDR positions is either not diversified, or it is diversified with a bias towards the following amino acid residues: Vkappa1: L55:Y, L94:L, L96:Y; Vlambda1: L96:Y; VH3: H50:T, H60:N, H63:I, H64:L, and H95:D; particularly wherein the listed amino acid residues is present to at least 30%, and more particularly to at least 50% in the diversification mixture; or
(ad) any of the following CDR positions is either not diversified or diversified with the indicated limited diversity only: Vkappa1: L55:YHW, L94:FHIKLRY, L96:FY; Vlambda1: L96:FY; VH3: H50:QT, H60:HNRS, H63:VIF, H64:KL, and H95:DNT.
wherein the antibody variable domain is selected from the group of:
(i) a Vkappa1 antibody variable domain having a sequence identity in the framework regions FR1 to FR3 of at least 90% to the antibody variable domain of SEQ ID No. 1, optionally comprising one or more of the following changes:
(b) in the framework regions:
(ba) L1:D to L1:A;
(bb) L2:I to L2:T; and/or
(bc) L70:D to L70:E;
(ii) a Vlambda1 antibody variable domain having a sequence identity in the framework regions FR1 to FR3 of at least 90% to the antibody variable domain of SEQ ID No. 2; and/or
(ii) a VH3 antibody variable domain having a sequence identity in the framework regions FR1 to FR3 of at least 90% to the antibody variable domain of SEQ ID No. 3, optionally comprising one or more of the following changes:
(b) in the framework regions:
(ba) H2:V to H2:A;
(bb) H37:V to H37:I;
(bc) H48:V to H48:I; and/or
(bd) H49:S to H49:G.

15. An antibody that has a melting temperature of above 95° C. when analysed by differential scanning calorimetry in pure 1× phosphate buffered saline pH 7.4 (containing 1.06 mM KH₂PO₄, 2.97 mM Na₂HPO₄×7 H₂O, 155.17 mM NaCl and no other supplements), using a scan-rate of 60° C. per hour, no gain and a scan range of 32° C. to 115° C.

16. The antibody of claim 15, wherein the melting temperature is above 100° C.
